(19) United States
(12) Patent Application Publication
(10) Pub. No.: US 2011/0203013 A1
(43) Pub. Date: Aug. 18, 2011

(54) DELIVERING COMPOSITIONS OF INTEREST TO PLANT CELLS

(75) Inventors: David J. Peterson, Ames, IA (US); William James Gordon-Kamm, Urbandale, IA (US)

(73) Assignee: PIONEER HI BRED INTERNATIONAL INC

(21) Appl. No.: 13/012,095

(22) Filed: Jan. 24, 2011

Related U.S. Application Data
(60) Provisional application No. 61/305,277, filed on Feb. 17, 2010.

(51) Int. Cl. A01H 1/06; C12N 15/82; C12N 5/04; C12N 5/10; B82Y 5/00

(52) U.S. Cl. 800/279; 435/468; 435/410; 435/412; 435/414; 435/415; 435/416; 800/281; 800/284; 800/290; 800/293; 435/418; 435/419; 977/773; 977/810; 977/762; 977/902; 977/795; 977/788; 977/838; 977/832

(57) ABSTRACT

Methods and mixtures for providing a composition of interest to a plant cell are provided. Methods using a mixture of particles are provided, wherein the mixture includes at least one nanoparticle associated with a microparticle via a lipid compound. Methods include methods for plant cell transformation, methods to increase transformation frequency, methods to increase gene targeting, and methods for plastid transformation. The nanoparticle comprises at least one composition of interest. Also provided are transformed plant cells, plants, and seeds produced using the methods and mixtures described herein. Further provided are the methods of making and using the mixtures of particles.

### TECHNICAL FIELD

The present invention relates to the use of a mixture of particles to provide a composition of interest to a plant cell.

### BACKGROUND

Plants have been transformed using a variety of methods, including bombardment of plant cells with dense microparticles carrying molecules of interest such as polynucleotides. Biolistic transformation methods can be used with essentially any plant species that can be cultured in vitro for stable and/or transient transformation, where Agrobacterium-mediated methods may have a more limited scope of target plants and/or tissues and are not typically used for transient transformation. Typically, polynucleotides are bound to the microparticles by precipitation of DNA using a chemical such as calcium chloride, and/or spermidine. This method may not be compatible for delivery of certain compositions of interest, for example, polynucleotides or other compositions such as polypeptides, subcellular organelles, microorganisms, or any combinations of these. A continuing need exists for methods to deliver a variety of compositions to plant tissues for transformation.

### SUMMARY

Methods and mixtures for providing a composition of interest to a plant cell are provided. Methods using a mixture of particles are provided, wherein the mixture includes at least one nanoparticle associated with a microparticle via a lipid compound. Methods include methods for plant cell transformation, methods to increase transformation frequency, methods to increase gene targeting, and methods for plastid transformation. The nanoparticle comprises at least one composition of interest. Also provided are transformed plant cells, plants, and seeds produced using the methods and mixtures described herein. Further provided are the methods of making and using the mixtures of particles.

### DETAILED DESCRIPTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present invention now will be described more fully hereinafter with reference to the accompanying examples, in which some, but not all embodiments are shown. Indeed, the invention may be embodied in many different forms and should not be construed as limited to the embodiments set forth herein; rather, these embodiments are provided so that this disclosure will satisfy applicable legal requirements.

Many modifications and other examples will come to mind to one skilled in the art having the benefit of the teachings presented herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The articles “a” and “an” are used herein to refer to one or more than one (i.e., to at least one) of the grammatical object of the article. By way of example, “an element” means one or more than one element.

Mixtures of particles and methods of using the mixtures of particles to deliver one or more compositions of interest to a plant cell are provided. The mixture comprises at least one nanoparticle associated with a microparticle via a lipid compound. The nanoparticle includes at least one composition of interest. Optionally, the nanoparticle-microparticle mixture comprises more than one composition of interest, wherein the compositions of interest may be associated with or loaded into or onto the nanoparticles, and/or microparticles. Provided herein are the methods of making and using such mixtures.

Additional compositions of interest can be associated with the nanoparticle or microparticle or both depending on the type of composition being delivered. Accordingly, several advantages conferred by the nanoparticle-microparticle mixtures include increasing the efficiency of delivery of nanoparticles, the ability to deliver a wide variety of compositions of interest to a plant cell in vivo or in vitro, and the ability to control the order and/or rate of release of the one or more compositions from the nanoparticle-microparticle mixture components. Exemplary compositions that may be delivered include, but are not limited to, an organic substance, an inorganic substance, a drug, a hormone, a hormone antagonist, an inducing agent, a polynucleotide, a polypeptide, a microorganism, a subcellular organelle, a growth factor, a polysaccharide, a vitamin, a messenger, a co-factor and the like. Various regions of the nanoparticle or microparticle or both may be used to deliver the compositions. Using these mixtures and methods may increase the efficiency of delivering compositions of interest into a plant cell compared to standard delivery techniques, e.g. bombardment using polynucleotides adsorbed to the surface of a nanoparticle or microparticle using calcium chloride and spermidine. In addition, the mixtures and/or methods can be used to deliver compositions of interest for modulating expression of a number of genes to affect phenotypic traits of the plant cell, increase transformation efficiency, facilitate target-specific genomic alterations including mutations, deletions, insertions, and/or site-specific integrations, plastid transformation, or delivery of factors that will not be integrated into a genome, such as helper proteins, inducing agents, cell proliferation factors, cell cycle regulators, hormones, hormone antagonists, nucleic acids, ligands, or other compounds.

The nanoparticle-microparticle mixture can be used to provide a composition of interest to a plant or plant cell. In some examples, upon delivery of the mixture of particles, a nanoparticle comprising a composition of interest may be taken up into various parts of cells. Examples of locations that a nanoparticle may be taken up into include without limitation cytosol, nucleus, tonoplasts, plastids, proplastids, etioplasts, chromoplasts, leucoplasts, elaioplasts, proteinoplasts, amyloplasts, chloroplasts, endoplasmic reticulum, mitochondria, cell wall, Golgi, vacuoles, lysosome, thylakoid, nucleolus, protein body, starch granule, cell membrane, lumen between a double-layer membrane. In some examples, the composition of interest is transiently provided to the target plant cell and/or tissue, or subcellular location wherein the composition of interest is not integrated into a genome. In other examples, the composition is taken up and stably integrated into a genome of the plant cell. In some examples, the composition of interest comprises a polynucleotide which is stably incorporated into the nuclear genome, a plastid genome, a chloroplast genome, and/or a mitochondrial genome.

Generally, any type of nanoparticle that can be associated with a microparticle and can carry a composition of interest may be used in the methods and mixtures. The terms associated and associating include any physical relationship by which the nanoparticles and microparticles in the mixture are co-delivered during any subsequent transfers or other physical manipulations. In some examples the nanoparticles are attached, or linked, to the microparticles. The association may be achieved using any suitable material and means, including chemical or physical linking or attachment by way of adsorption or chemical conjugation. For example, the attachment or linkage may be by means of covalent bonding, ionic bonding, or non-covalent bonding, such as van der Waals forces, hydrogen bonding, adsorption, absorption, metallic bonding, or any combination thereof.

Any lipid compound that will attach or associate the nanoparticle to the microparticles, and/or a composition of interest to the nanoparticle, the microparticle, or the mixture of particles can be used. In some examples the lipid compound is a polyelectrolyte, polyampholyte, fatty acid, neutral lipid, lipid solutions, cationic lipid solution, a liposome solution, a ionic polymer, a anionic polymer, a cationic polymer, a protein, a DNA binding protein, a cationic protein, a cationic peptide, surfactant, detergent, polyamino acid, or a cationic polyamino acid. Polyelectrolytes are polymers whose repeating units bear an electrolyte group. These groups will dissociate in aqueous solutions, making the polymers charged. Polyelectrolyte properties are similar to both electrolytes (salts) and polymers. Many biological molecules are polyelectrolytes, for example polypeptides and polynucleotides DNA, and synthetic polyelectrolytes are widely available. Polyelectrolytes which bear both cationic and anionic repeat groups are called polyampholytes. A fatty acid is a carboxylic acid or organic acid, often with a long aliphatic tail, and includes saturated and unsaturated molecules. Fatty acids typically include chains as short as butyric acid (4 carbons). Fatty acids derived from natural fats and oils typically have at least 8 carbon atoms, e.g. caprylic acid (octanoic acid). Fatty acids can be synthesized by the hydrolysis of the ester linkages in a triglyceride, with the removal of glycerol. Cationic lipids have a net positive charge, and many are available for transfection of mammalian cells with polynucleotides and/or polypeptides. The transfection solution sometimes has a second lipid component, such as a neutral or fusogenic lipid, to facilitate uptake across the cell membrane. Many cationic lipids are commercially available including: 293fectin™ which comprises a proprietary cationic lipid based formula optimized for use with 293 cells; Cellfectin® comprising a cationic lipid 1:1.5 (M/M) liposome formulation of cationic lipid N, NI, NII, NIII-Tetramethyl-N,NI,NII,NIII-tetrapalmityl-spermine (TM-TPS), and dioleoyl phosphatidylethanolamine (DOPE); DMRIE-C comprising a cationic lipid 1:1 (M/M) liposome formulation of cationic lipid DMRIE 1,2-dimyristyloxy-propyl-3-dimethyl-hydroxy ethyl ammonium bromide and cholesterol; Freestyle™ MAX comprising a proprietary cationic lipid designed for Freestyle™ cells; Lipofectamine™ comprising a 3:1 (w/w) liposome formulation of polycationic lipid 2,3-dioleyloxy-N-[2(sperminecarboxamido)ethyl]-N,N-dimethyl-1-propanaminium trifluoroacetate (DOSPA) and neutral lipid dioleoyl phosphatidylethanolamine (DOPE) (all of which are available from InVitrogen); Tfx™ reagents (TFX-10, TFX-20, TFX-50) all contain the same concentration of the cationic lipid component with different molar ratios of the fusogenic lipid, comprising a mixture of a synthetic, cationic lipid molecule [N,N,N′,N′-tetramethyl-N,N″-bis(2-hydroxyethyl)-2,3-di(oleoyloxy)-1,4-butanediammonium iodide] and L-dioleoyl phosphatidylethanolamine (DOPE); TransFast™ comprising the synthetic cationic lipid, (+)-N,N [bis(2-hydroxyethyl)]-N-methyl-N-[2,3-di(tetradecanoyloxy)propyl]ammonium iodide and the neutral lipid, DOPE; Transfectam® comprises a synthetic, cationic lipopolyamine molecule dioctadecylamidoglycyl spermine (DOGS) with the spermine group is covalently attached through a peptide bond to the lipid moiety (all available from Promega); CLONfectin™ comprises a cationic, amphiphilic lipid used for liposome-mediated transfection of mammalian cells (available from CloneTech); ESCORT™ liposome transfection reagent comprises 1:1 (w/w) liposome formulation of the cationic lipid N-[1-(2,3-dioleoyloxy)propyl]-N,N,N-trimethylammonium chloride (DOTAP) and dioleoyl phosphatidylethanolamine (DOPE); ESCORT II is a unique formulation of the neutral lipid dioleoyl phosphatidylethanolamine (DOPE) and a proprietary cationic lipid; and DOTAP methosulfate comprising N-(2,3-dioleoyloxy-1-propyl)trimethylammonium methyl sulfate cationic liposome-forming compound (all available from Sigma Chemical Co.). Other compounds include hexadimethrine bromide 1,5-dimethyl-1,5-diazaundecamethylene polymethobromide (Kawai et al. (1984) Mol Cell Biol 4:1172-1174); poly-L-ornithine/DMSO; polybrene/DMSO; polybrene/glycerol; polyethyleneimine (PEI), chitosan, protamine CI, DNA binding proteins, histone H1, histone CENH3, poly-L lysine, DMSA, and the like.

In some examples, the mixtures of particles for direct delivery are prepared by associating the nanoparticles with the microparticles and/or the composition(s) of interest in the presence of Tfx-10™, Tfx-20™, Tfx-50™, Lipofectin™ Lipofectamine™, Cellfectin™, Effectene™, Cytofectin GSV™, Perfect Lipids™ DOTAP, DMRIE-C, FuGENE-6™, Superfect™, Polyfect™, polyethyleneimine (PEI), chitosan, protamine CI, DNA binding proteins, histone H1, histone CENH3, poly-L lysine, DMSA, and the like. In some examples the lipid compound is a cationic lipid solution comprising N,N,N′,N′-tetramethyl-N,N′-bis(2-hydroxylethyl)-2,3-di(oleoyloxy)-1,4-butanediammonium iodide. In some examples the cationic lipid solution further comprises L-dioleoyl phosphatidylethanolamine (DOPE).

Exemplary nanoparticles include but are not limited to ceramic nanoparticles, gold nanoparticles, gold-coated nanoparticles, porous nanoparticles, mesoporous nanoparticles, silica nanoparticles, mesoporous silicates polymer nanoparticles, tungsten nanoparticles, gelatin nanoparticles, metal nanoparticles, metal oxide nanoparticles, metal-nonoxide nanoparticles, organic nanoparticles, biomolecular nanoparticles, nanotubes, nanoshells, nanocores, nanospheres, nanorods, magnetic nanoparticles, quantum dots (nanocrystals) or combinations thereof. See, for example, published applications US2006/0154069, US2006/0018966, and WO2009/046384, herein incorporated in their entirety. Generally, the nanoparticles are non-toxic to a plant cell. Nanoparticles may be prepared according to known methods (see, for example, Tomalia et al. (1990) Chem Int Ed Engl 29:5305; and US2006/0154069, US20060018966, WO2009/040811, WO2009/040553, WO 2009/040553, WO2009/038659, WO2009/035657, WO2009/026376, and WO2009/012303, herein incorporated in their entirety).

A suitable nanoparticle may be of any shape or size provided that it can be associated with the microparticle(s) and carry a composition of interest. In some examples the nanoparticle is essentially spherical, polygonal, rod-shaped, or irregular in shape. Any size of nanoparticle may be used make a nanoparticle-microparticle mixture to provide a composition of interest to a plant cell. Typical sizes of nanoparticles include without limitation about 0.01 to about 1000 nanometers (nm). Most commonly used nanoparticles are about 1 nm to about 100 nm in diameter. Examples of useful nanoparticle sizes include about 0.01, 0.05, 0.1, 0.5, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 or more nanometers in diameter.

Microparticles include any solid carrier used for delivery of a composition of interest into the interior of a cell. A microparticle should be of sufficient mass so that when it is accelerated it is able to penetrate the target cell or tissue. Any microparticle can be used, examples of microparticles include but are not limited to metal particles such as gold, tungsten, palladium, rhodium, platinum or iridium particles used for particle bombardment of cells, and gold nanoparticles used in cellular uptake; whiskers such as alumina, silica, glass, ceramic, titania, zirconia, boron, carbon, carbides, silicon carbide whiskers; carbon nanofibers, optionally arrayed as vertically aligned carbon nanofiber (VACNFs) arrays; and nanomaterials such as mesoporous silicate, and the like. Microparticles include solid microparticles that may be naked or uncoated prior to contact with the lipid compound and nanoparticle or composition of interest.

Microparticles used for particle bombardment are typically made from metals such as gold or tungsten, and range in size from about 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, or 2.0 μm. Microparticles for use for whiskers-mediated transformation are typically made of silicon carbide, and range in length from about 4 μm, 5 μm, 6 μm, 7 μm, 8 μm, 9 μm, 10 μm, 12 μm, 15 μm, 20 μm, 25 μm 30 μm, or 40 μm, and range in width from 0.2 μm, 0.3 μm, 0.4 μm, 0.5 μm, 0.6 μm, 0.7 μm, 0.8 μm, 0.9 μm, or 1.0 μm with commonly used whiskers including, for example 30 μm×0.5 μm and 10 μm×0.3 μm.

The nanoparticle-microparticle mixture may comprise any nanoparticle associated with microparticle available in the art. The mixture may comprise more than one type of nanoparticle and/or microparticle. Further, the ratio of nanoparticles and microparticles in the mixture may be varied as needed. In some examples the mixture comprises one type of nanoparticle combined with one type of microparticle, wherein the nanoparticle and/or the microparticle comprise a composition of interest. In other examples the mixture comprises more than one type or size of nanoparticle combined with more than one type or size of microparticle, where at least one type of nanoparticle and/or microparticle comprises at least one composition of interest. In some examples the ratio of nanoparticles and microparticles in the mixture can range from about 10⁵ total nanoparticles to 1 microparticle (10⁵:1), to 1 nanoparticle to about 10⁵ total microparticles (1:10⁵). For example, useful nanoparticle:microparticle ratios include mixtures having about 10⁵:1, 10⁴:1, 10³:1; 10²:1, 1:1, 1:10², 1:10³, 1:10⁴, and 1:10⁵, based on estimated particle counts. In other examples the nanoparticle:microparticle can be described by the mass of each particle in the mixture. For example, the nanoparticle:microparticle ratio in the mixture can range from about 10⁻² ng nanoparticles to 10⁵ ng microparticles (10⁻²:10⁵) to about 10⁵ ng nanoparticles to 10⁻² ng microparticles (10⁵:10⁻²). For example, useful nanoparticle:microparticle ratios include mixtures having about 10⁻²:10⁵, 10⁻²:10⁴, 10⁻²:10³, 10⁻²:10², 10⁻²:10, 10⁻²:1, 10⁻²:10⁻¹, 10⁻²:10⁻², 10⁻¹:10⁵, 10⁻¹:10⁴, 10⁻¹:10³, 10⁻¹:10², 10⁻¹:10, 10⁻¹:1, 10⁻¹:10⁻¹, 10⁻¹:10⁻², 1:10⁵, 1:10⁴, 1:10³, 1:10², 1:10, 1:1, 1:10⁻¹, 1:10⁻², 10:10⁵, 10:10⁴, 10:10³, 10:10², 10:10, 10:1, 10:10⁻¹, 10:10⁻², 10²:10⁵, 10²:10⁴, 10²:10³, 10²:10², 10²:10, 10²:1, 10²:10⁻¹, 10²:10⁻², 10³:10⁵, 10³:10⁴, 10³:10³, 10³:10², 10³:10, 10³:1, 10³:10⁻¹, 10³:10⁻², 10⁴:10⁵, 10⁴:10⁴, 10⁴:10³, 10⁴:10², 10⁴:10, 10⁴:1, 10⁴:10⁻¹, 10⁴:10⁻², 10⁵:10⁵, 10⁵:10⁴, 10⁵:10³, 10⁵:10², 10⁵:10, 10⁵:1, 10⁵:10⁻¹, and 10⁵:10⁻² ng nanoparticles:ng microparticles. The ratio of nanoparticles:microparticles in the mixture can also be described as a percentage of the total mixture. For example, the nanoparticle:microparticle ratio in the mixture can range from about 99.9% nanoparticles to about 0.1% (99.9:0.1) microparticles to about 0.1% nanoparticles to about 99.9% microparticles (0.1:99.9) based on the weight of the particles (w/w percent). For example, useful nanoparticle:microparticle ratios include mixtures having about 99.9:0.1, 99.5:0.5, 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, 1:99, 0.5:99.5, and about 0.1:99.9 w/w percent.

The nanoparticle may be made from any material, or combination of materials, with various properties. The nanoparticle may include a gel, a solid, a polymer, a metal, a ceramic, a glass-ceramic, and/or a silicate, and may be non-porous or porous having one or more pores, or any combinations thereof. The size and material used to synthesize the nanoparticle may selected based on the intended use, for example, the final particle size desired, the surface properties (such as charge), chemical compatibility, the inclusion of pores, the size of the pores, the ratio of size to surface area desired, the weight or mass of the nanoparticle, the chemical properties of the nanoparticle, the composition of interest to be delivered and how the composition of interest is to be carried, e.g. within the nanoparticle, on the exterior of the nanoparticle, incorporated into the nanoparticle, or a combination of any these criteria. Materials and techniques suitable for preparing nanoparticles will be known to one skilled in the art and are also described elsewhere herein. Nanoparticles suitable for the present methods and mixtures are commercially available. For example, silica spheres are commercially available, for example, from Polysciences, Inc. (Warrington, Pa.) and Fiber Optic Center, Inc. (Bedford, Mass.) to name a few companies.

The nanoparticle may be comprised of one or more regions. These regions may include but are not limited to a core, a pore, and/or one or more layers, or combinations thereof. See for example WO2006/084339 incorporated by reference in its entirety. The core, pore, or layer or combinations thereof may be composed of the same or different materials depending on the intended use of the nanoparticle, the composition to be delivered and the desired sequence and rate of release of the composition. For example, the nanoparticle core, pore or layers may be formed from any suitable material, including without limitation, polymers, for example, including but not limited to, ceramic, hydrolyzed silane, for example a hydrolyzed alkoxysilane, and may include silica and/or a polysilsesquioxane polylactide, polyglycolide and copolymers of the aforementioned polymers (commonly known as poly lactic glycolic acids or PLGA), poly aminoacids, copolymers of polyaminoacids, glycosamino glycans, lipidated glycosaminoglycans and the like. See, for example, US2006/0154069 and US2006/0018966, herein incorporated by reference in their entirety. The material may be selected to achieve the desired rate and sequence of release of the composition(s) of interest associated with the nanoparticle. Likewise, the core, a pore, or layers or combinations thereof may be similar or dissimilar in release rate or decomposition rate. In some examples, a composition of interest is associated with the core, or one or more layers, and the composition is released when the core or layers or both are degraded. For example, high molecular weight PLGA polymers may be used when a slower release is desired, and the low molecular weight variants of PLGA selected when a more rapid release of the composition is desired. See WO2009/036253. The ratio of low and high molecular weight polymers such as PLGA, can be altered so as to regulate the release of any incorporated composition.

In some examples, the core is used to deliver a composition of interest. When the core is to be used for delivery of the composition, the core should have a size, shape and material suitable to carry the desired amount and kind of composition of interest. The size of the core may correlate to the size of nanoparticle described above, for example, having a size from 0.01 to about 1000 or more nanometers in diameter. The core may have any suitable shape, such as spherical, polygonal, non-spherical, for example, rod-shaped, or irregular, so long as the core functions for its intended purpose. By way of example and not limitation, the core may be a gel, solid, non-porous, porous having one or more pores, microporous having one or more pores, mesoporous, or combinations thereof.

In some examples, a composition of interest is loaded within or associated with the interior of the core. For example, the composition of interest may be encapsulated or incorporated into the core, and/or may be partially or fully throughout the core. The composition of interest may be incorporated into the core of the nanoparticle, e.g. substantially homogenously or in a heterogeneous matter. The composition of interest may be associated with the core in any suitable manner, for example, by way of chemical and/or physical linking or attachment by way of adsorption or chemical conjugation. For example, the composition may be conjugated to a polymer within the nanoparticle core, such as a non-charged small drug attached to larger molecules, in some cases these are charged polymers. If degradation of the composition of interest is of concern, the composition may be associated with a region of the nanoparticle, e.g. the core or a capped pore, where the composition may be protected from degradation and/or inactivation that it retains its biological activity or effectiveness until it is released from the nanoparticle.

In some examples, the nanoparticle includes one or more layers that surround the core. When one or more layers is to be used for delivery of a composition at least one of the layers should have a size, shape and material suitable to deliver the desired amount and kind of composition of interest. The layer(s) may have any suitable shape, such as spherical or non-spherical, for example, rod-shaped so long as the one or more layers functions for its intended purpose. For example, the one or more layers may be a gel or a solid, and may be non-porous, porous microporous, or mesoporous, or a combination thereof.

In some examples, a composition of interest is associated with the one or more layers. In some examples the composition of interest may be encapsulated, or incorporated into one or more of the layers, for example it may be partially or fully dispersed throughout one or more of the layers. The composition of interest may be incorporated into one or more layers of the nanoparticle substantially homogenously, or in a heterogeneous matter. The composition of interest, such as a drug or hormone, may be incorporated into one or more of the layers in any suitable manner, for example, by way of chemical and/or physical linking or attachment by way of adsorption or chemical conjugation. For example, the composition may be conjugated to a polymer within one or more of the layers, such as a non-charged small drug attached to larger molecules, typically charged polymers. If degradation of the composition of interest is of concern, the composition may be loaded into regions of the nanoparticle where it may be protected from degradation and/or inactivation so that it retains its biological activity or effectiveness until it is released from the nanoparticle. The nanoparticle has an outer layer, which in some cases is the exterior surface of the core, to which a composition of interest may be associated for delivery. In some examples, the composition of interest is associated with an interior and/or an exterior surface of any one or more of the regions of the nanoparticle.

In some examples, the nanoparticle has one or more pores which may be loaded with a composition of interest. The pore may be of any size, shape, structure. For loading and delivery of a composition of interest the pore should have a size, shape, structure and chemical properties suitable to carry the desired type and amount of a composition of interest. A porous nanoparticle may have pores with a diameter of from about 1 to about 100 nm or greater. Typically, a microporous nanoparticle has pores with a diameter less than about 2 nm. Mesoporous particles generally have pores with a diameter of about 2 nm to about 100 nm. For example, a mesoporous particle has pores of about 3, 4 5, 6, 7, 8, 9, 10, 11, 12, 13, 14, 15, 16, 17, 18, 19, 20, 25, 30, 35, 40, 45, 50, 55, 60, 65, 70, 75, 80, 90, or about 100 nm in diameter. In some cases, the nanoparticle pores are microporous or mesoporous or a combination thereof. The pores may be uniform or variable in size and shape. The pore may be composed of any suitable material, including materials used to form a core and/or a layer of the particle. In some examples, the composition is loaded within one or more pores and the composition or pore or nanoparticle modified to prevent the premature release of the composition. See, for example, WO2005/009602, US2006/0154069, and US2006/0018966 herein incorporated by reference in their entirety.

In some examples where the pore is loaded with a composition of interest and if the composition and pore do not associate naturally, the pore may be modified so that an association with the loaded composition of interest and pore can be made (see, e.g., U.S. Pat. No. 6,303,179, herein incorporated by reference in its entirety). The composition, pore or surface of the nanoparticle may be modified so that it includes a labile group, a charge, a ligand, a co-factor, a pH-sensitive bond, a temperature-labile bond, a photochemically active group, a functional group including an amide (e.g, amidoamine, for example a poly (amidoamine) chain), a peptide bond, an amine, a thiol (such as a disulfide bond), a carboxyl group, an ester group or bond, is hydrophilic, is hydrophobic, is amphipathic, is polar, is non-polar, and the like. In one aspect, the functional group is a reactive group or bond. The labile group may be associated with any part of the nanoparticle and/or the composition through covalent bonding, non-covalent bonding, ionic bonding, non-ionic bonding, hydrogen bonding, or any other interaction. In some cases, the labile group may be biodegradable, for example, a poly (amidoamine) chain. Any suitable technique or materials for modifying the nanoparticle or portion therefore (e.g., core, layer, and/or pore) to comprise a labile group may be used. In some examples, a labile group will be part of or associated with a linker so that the composition of interest may be attached to the nanoparticle to prevent the premature release of the composition. Any suitable linker may be used, for example, a polymer such as an amide-functional polymer. The linker may be associated with the nanoparticle using any suitable technique, for example, via a labile group, the material of a pore, the core and/or a layer.

In one example, the composition is released from the pore following delivery to a target cell or tissue. The composition of interest may be released from the pore by exposing the labile group to conditions that disrupt the association between the labile group and the composition of interest. Exemplary conditions include exposure to an environment with a pH that disrupts a pH-sensitive bond, a chemical that disrupts a bond (e.g., disrupting a disulfide bond via a reducing agent such as DTT), an enzyme that disrupts a peptide bond, a temperature that disrupts a temperature-sensitive bond, or the appropriate wavelength(s) of light to disrupt a photochemically active group or bond (see WO2005/009602 incorporated by reference in its entirety). The conditions may disrupt the labile group so that the composition of interest is released. In some cases, the conditions may selectively disrupt the labile group. The condition may naturally exist at the target cell or tissue where it is desirable to release the composition. In other examples the condition can be created at a time and place where the release of the composition is desired, e.g. in a plant cell culture via addition of agents or changing conditions.

Employing a nanoparticle with a pore allows for a larger quantity of one or more compositions of interest to be delivered by the nanoparticle. It also provides means for controlling the timing, place, and/or release of one or more compositions of interest, including controlling the sequence of release of two or more compositions of interest, which may be also differentiated by the cellular or subcellular localization of the nanoparticle at the time of release of a first composition of interest as compared to release of a subsequent composition of interest. In some examples, a microparticle in the nanoparticle-microparticle mixture also comprises a composition of interest. In some examples release of the composition of interest from the microparticle takes place at a different time and/or location than release of a 2^{nd} composition of interest from the non-porous or porous nanoparticle. In some examples, using a porous nanoparticle provides a means to deliver a combination of compositions of interest, which in some instances are chemically incompatible with each other at one or more phases of the particle preparation, mixture preparation, composition loading, and/or delivery processes.

When the composition of interest and pore do not associate naturally, the composition may be loaded within the pore and the pore capped with a material to prevent premature release the composition of interest (see WO2005/009602, US2006/0154069, and US2006/0018966 incorporated by reference in their entirety). Any suitable cap that prevents the composition's premature release may be used. For example, the cap can be covalently bonded to the nanoparticle or it can be associated thereto through ionic, hydrogen bonding, or other interactions. The cap can be positioned in the opening of a pore or it can be located within the pore itself (e.g., bonded or linked to the interior surface of the pore). The cap can be a discrete body, such as a particle or crystal of an inorganic salt, protein, or a polymeric nanosphere, or it can be in the form of a polymer coating that partially or completely covers the pore and/or particle. Any suitable materials or techniques to achieve this end may be used. Exemplary cap materials and/or forms include without limitation: polymers, crystalline, coatings, layers, nanoparticles, nanospheres, nanorods, quantum dots, and the like, comprising materials including but not limited to a metal, a gold nanoparticle, a sulfide salt nanoparticle, a cadmium sulfide nanoparticle, inorganic particles, metal oxides, metal sulfides, inorganic molecules, inorganic crystals, organic molecules, biodegradable oligomers, dendritic polymers (dendrimers), poly (amidoamine), poly (lactic acid), hyper-branched polymer, anionic dendrimers, cationic dendrimers, a biopolymer, an organic polymer, a peptide, a polypeptide, a protein, an oligonucleotide, a polynucleotide, an oligosaccharide, or a polysaccharide or combinations thereof. In some cases, a coating that partially or completely covers the nanoparticle or pore, such as a polymer coating, may be used as a cap. In some aspects, the caps are biodegradable. The cap may block the pore so that the composition is not released until the cap is removed. The cap may be associated with the nanoparticle at any suitable manner. For example, the cap may be associated with an interior or exterior surface of the pore, and/or associated with an interior or exterior surface of the nanoparticle core, and/or layer, e.g., adjacent to the pore. In some examples the cap can be chosen in order to confer a function or property other than simply preventing or slowing the release of the composition of interest.

In various aspects the mixture comprising the associated microparticles and nanoparticles may also include any non-toxic carrier(s). The carrier, may comprise water, a buffer (e.g., saline, glycine, histidine, glutamate, succinate, phosphate, acetate, aspartate, a Good's buffer), a cell culture media, alcohol solutions (e.g, ethanol), proteins, blocking agents, polynucleotides, sugars, stabilizers, salts, and the like or combinations thereof.

As discussed above, any region or combination of regions of the nanoparticle and microparticle may be used to facilitate the delivery of one or more compositions of interest. For example, the composition may be associated with the surface of the nanoparticle, the surface of the microparticle, incorporated into the nanoparticle core, a nanoparticle, or nanoparticle pore and any combinations thereof.

The particle mixtures and methods may be employed to deliver various compositions of interest to a plant cell. In some examples, the composition of interest is bioactive. A bioactive composition of interest includes any composition that has and retains biological activity. Non-limiting examples of compositions of interest may include an organic substance, an inorganic substance, a polynucleotide, an oligonucleotide, a chimeric oligonucleotide, a peptide, a polypeptide, a peptide nucleic acid, a drug, a microorganism, a subcellular organelle, an antibiotic, a selection agent, a herbicide, a fungicide, an antiviral agent, a growth factor, a hormone, a hormone antagonist, a polysaccharide, a lipid, a ligand, a metabolite, a vitamin, an inducer, and the like or any combinations thereof. In some instances, the composition of interest may be characterized in more than one of the above exemplary categories.

The composition(s) of interest may be released from the particle mixture in a variety of means, and combinations thereof. In some examples the composition(s) of interest passively dissociate from the mixture, in some examples the composition(s) of interest are released via cleavage of a bond, including chemical or biochemical cleavage, in some examples the composition(s) of interest are released by degradation of a linker, bond, or moiety,

In some examples an effective amount of a composition of interest is delivered to a plant cell. An effective amount of any composition of interest is that which is sufficient to achieve the desired response. For example, an effective amount of an enzyme is any amount in a range sufficient to detect enzyme activity, reaction product, and/or phenotype in a recipient plant cell. In another example, an effective amount of a polynucleotide is any amount in a range sufficient to detect a phenotype in a target cell, including but not limited to detecting an expression product (DNA, RNA, and/or protein), detecting an integration event (e.g., operable linkages, flanking sequence, and/or gene targeting), and/or detecting changes in cell properties (e.g., growth rate, morphology, differentiation, dedifferentiation, etc.). The exact amount required will vary depending on the particular composition of interest, the mode of delivery, the release mechanism, the kinetics and/or timing of release, the type of plant, the type of plant cell, genotype, the subcellular localization, any co-factors (endogenous or exogenous), and the desired effect. An appropriate effective amount of any specific composition of interest may be determined by one of ordinary skill in the art using routine experimentation.

In some examples, the composition of interest to be delivered is a chemical, for example, a drug, a ligand, an inducer, a hormone, or hormone antagonist. A drug is any agent to prevent, treat, or promote a condition. A drug may be a chemical, or peptide, or an antibiotic such as an aminoglycoside, gentamycin, neomycin, carbenicillin, spectinomycin, rifampicin, or streptomycin. The composition of interest may include one or more hormones, for example, a plant hormone. Hormones may be synthetic or natural or any combination thereof. Exemplary plant hormones or hormone antagonists include but are not limited to auxins such as indole acetic acid, naphthalene acetic acid, dicamba, and 2,4-D, anti-auxins such as 2,4,6-trichlorobenzoic acid or 2-(2,4-dichlorophenoxy) proprionic acid, gibberellins such as gibberellic acid, cytokinins such as zeatin, kinetin, thidiazuron or benzylaminopurine, abscisic acid, ABA inhibitors such as aminotriazole, ethylene, ethylene-substitutes such as 1-propene, competitive inhibitors of ethylene such as 1-butene, 1-pentene, 1-hexane, 1-octene, 1-decene, 1-dodecene, or ethylene blockers such as norbornadiene.

In some examples, chemical that may reduce bombardment-mediated stress response may be introduced. For example chemicals that inhibit ethylene action (e.g., 1-butene, 1-pentene, 1-hexane, 1-octene, 1-decene, 1-dodecene, or norbornadiene) may be provided during bombardment. Some of these chemicals have health & safety issues regarding human exposure. This provides a means to safely load these compounds, minimizing human exposure. These mixtures and methods also provide a means to provide chemicals that are not typically compatible or soluble in media, or that have the potential to modify the media or a media component, or which may not be uniformly distributed in an aqueous media solution. The particle mixtures and methods also provide a means for direct delivery of known amounts of compounds to cells directly impacted by the transformation process rather than indirectly providing compounds to the whole culture and having issues of unknown or unmeasurable uptake of chemical by whole culture, with unknown amounts delivered to target cells. In other examples, an ABA-mediated stress response could be modulated by introducing norflurazone.

In some examples, the mixtures and methods can be used to deliver auxins and/or cytokinins to cells to stimulate growth. Delivery by these methods is expected to provide a transient growth pulse to help cells survive the potentially negative impacts of the initial harvesting of explants, culture, bombardment, and the like. In a similar way, the mixtures and methods can be used to deliver cell proliferation factors including polypeptides that stimulate cell growth such as knotted, wuschel, babyboom, and cyclins.

In some examples, the mixtures and methods could be used to pre-treat or treat plant cells and/or tissues to enhance transformation by another means. For example, the mixtures could be used to wound tissues for Agrobacterium-mediated transformation. Optionally, the particle mixture, e.g., nanoparticles, could be loaded with acetosyringone, which may enhance cell response to Agrobacterium-mediated transformation. In some examples, the mixture may comprise one of more of the virulence or cytokinin-stimulating genes, RNAs, or polypeptides which may also enhance cell response to Agrobacterium-mediated transformation.

The composition of interest to be delivered may include one or more polynucleotides. A polynucleotide is any nucleic acid molecule polymer, including DNA, RNA, DNA:RNA chimeras, peptide nucleic acids (PNA), and any combination thereof. Polynucleotides include naturally occurring molecules isolated from any suitable source (e.g., plant, virus, prokaryote, eukaryote), and/or synthetic or modified molecules including those having modified bases (e.g., ribonucleotides, deoxyribonucleotides), and any combination thereof. Polynucleotides suitable for use in the mixtures and methods described herein may be endogenous, or may be heterologous to the plant cell, plant, organelle, or target site sequence to be transformed. Heterologous polynucleotides are from a different source, or at a different locus (e.g., have different flanking sequences) than found in nature. Polynucleotides encompass all forms including, but not limited to, single-stranded, double-stranded, triplexes, linear, circular, branched, hairpins, stem-loop structures, branched structures, and the like. The polynucleotides may be of any size, form and/or composition suitable to achieve the desired result. The polynucleotides can be any size from short oligonucleotides typically less than about 20 nucleotides (nt) in length, oligonucleotides typically less than 120 nt, small polynucleotides typically about 120 nt to about 2 kb, moderately sized polynucleotides typically greater than about 2 kb to about 20 kb, large polynucleotides typically greater than about 20 kb to about 75 kb, and very large polynucleotides greater than 75 kb.

In some examples the composition of interest includes a polynucleotide. When the polynucleotide is a DNA construct, it may be generated using any molecular biological technique or synthetic technique to juxtapose at least two heterologous nucleic acid sequences in a polynucleotide. Accordingly, in some instances, the composition of interest includes at least one DNA construct. A DNA construct comprises a polynucleotide which when present in the genome of a plant is heterologous or foreign to that chromosomal location in the plant genome. In preparing the DNA construct, various fragments may be manipulated to provide the sequences in a proper orientation and/or in the proper reading frame. Adapters or linkers may be employed to join the fragments. Other manipulations may be used to provide convenient restriction sites, removal of superfluous DNA, or removal of restriction sites. For example, in vitro mutagenesis, primer repair, restriction, annealing, resubstitutions, transitions, transversions, or recombination systems may be used. Any nucleic acid element may be included in the DNA construct(s) for any purpose, including but not limited to untranslated regions, regulatory regions, transcription initiation regions, translation initiation regions, introns, exons, polynucleotides encoding an RNA, protein coding regions, markers, recombination sites, target sites, restriction sites, recognition sites, insulators, enhancers, spacer/stuffer sequences, origins of replication, telomeric sequence, centromeric sequences and/or repeats, diagnostic sequences, operators, and the like, can be provided in a DNA construct(s). The construct can include 5′ and 3′ regulatory sequences operably linked to the appropriate sequences. The DNA construct(s) can include in the 5′ to 3′ direction of transcription at least one of the following, a transcriptional and translational initiation region, the polynucleotide, and a transcriptional and translational termination region functional in plants. Alternatively, the DNA construct(s) may lack at least one 5′ and/or 3′ regulatory element. In some examples DNA construct(s) are designed such that upon introduction into a cell and in the presence of the appropriate recombinase a recombination event at the target site operably links the 5′ and/or 3′ regulatory regions to the appropriate sequences of the DNA construct(s). Operably linked means that the nucleic acid sequences linked are contiguous and comprise a functional linkage of the components. In some examples intervening sequences can be present between operably linked elements and not disrupt the functional linkage. For example, an operable linkage between a promoter and a polynucleotide of interest allows the promoter to initiate and mediate transcription of the polynucleotide of interest. In some examples a translational start site is operably linked to a recombination site. In some examples, a recombination site is within an intron. The cassette may additionally contain at least one additional sequence to be introduced into the plant. Alternatively, additional sequence(s) can be provided separately. DNA constructs can be provided with a plurality of restriction sites or recombination sites for manipulation of the various components and elements. DNA constructs may additionally contain selectable marker genes. Where appropriate, polynucleotides may be modified for increased expression in the transformed plant. For example, the polynucleotides can be synthesized using plant-preferred codons for improved expression. see, e.g., Campbell & Gowri (1990) Plant Physiol 92:1-11. Additional sequence modifications can enhance gene expression in a cellular host including elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. The sequence may also be modified to avoid predicted hairpin secondary mRNA structures.

A promoter is a region of DNA involved in recognition and binding of RNA polymerase and other proteins to initiate transcription. A plant promoter is a promoter capable of initiating transcription in a plant cell, for a review of plant promoters see Potenza et al. (2004) In Vitro Cell Dev Biol 40:1-22. The promoter may be any promoter suitable for expression, for example, constitutive, developmental stage-preferred, tissue-preferred, and/or inducible promoters including chemical, light, metal, repressible or other inducible or regulated systems. Chemical-inducible promoters are known and include, but are not limited to, the maize In2-2 promoter, which is activated by safeners (De Veylder et al. (1997) Plant Cell Physiol 38:568-77), the maize GST promoter (GST-II-27, WO93/01294), which is activated by select compounds used as pre-emergent herbicides, the PR-1 promoter (Cao et al. (2006) Plant Cell Rep 6:554-60), which is activated by BTH or benxo(1,2,3)thiaidazole-7-carbothioic acid s-methyl ester, the tobacco PR-1a promoter (Ono et al. (2004) Biosci Biotechnol Biochem 68:803-7), which is activated by salicylic acid, the copper inducible ACE1 promoter (Mett et al. (1993) PNAS 90:4567-4571), the ethanol-inducible promoter AIcA (Caddick et al. (1988) Nature Biotechnol 16:177-80), an estradiol-inducible promoter (Bruce et al. (2000) Plant Cell 12:65-79), the XVE estradiol-inducible promoter (Zao et al. (2000) Plant J 24:265-273), the VGE methoxyfenozide inducible promoter (Padidam et al. (2003) Transgenic Res 12:101-109), the TGV dexamethasone-inducible promoter (Bohner et al. (1999) Plant J 19:87-95), steroid-responsive promoters such as the glucocorticoid-inducible promoter (Schena et al. (1991) PNAS 88:10421-10425, and McNellis et al. (1998) Plant J. 14:247-257) and tetracycline-inducible and tetracycline-repressible promoters (Gatz et al. (1991) Mol Gen Genet. 227:229-237; Gatz et al. (1992) Plant J 2:397-404; and U.S. Pat. Nos. 5,814,618 and 5,789,156), herein incorporated by reference. Suitable promoters to achieve the desired level, temporal and spatial expression for the operably linked polynucleotide of interest will be apparent to those skilled in the art.

The polynucleotide may include or encode an RNA of interest, which may be expressed in the cell. In some examples the expressed RNA (e.g., mRNA) is translated into a protein. In some examples, the RNA is the active agent, e.g., an rRNA, a tRNA, a ribozyme, or an RNAi agent. An RNAi agent is any RNA involved in RNA interference and/or reduces expression of a target molecule, including but not limited to a double-stranded RNA, miRNA precursor, a miRNA, a sRNA precursor, a sRNA, a transacting sRNA precursor, a transacting sRNA, an RNAi precursor, an antisense polynucleotide precursor, an antisense polynucleotide, a sense-suppression precursor, a sense-suppression polynucleotide, or a ribozyme. Other polynucleotides and methods for inhibiting or eliminating the expression of a gene in a plant cell, a plant, a plant pathogen, and/or plant pest are well known (see, e.g., Sheehy et al. (1988) Proc Natl Acad Sci USA 85:8805-8809; and U.S. Pat. Nos. 5,107,065; 5,453,566; and 5,759,829); RNA interference (Napoli et al. (1990) Plant Cell 2:279-289; U.S. Pat. No. 5,034,323; Sharp (1999) Genes Dev 13:139-141; Zamore et al. (2000) Cell 101:25-33; Javier (2003) Nature 425:257-263; and, Montgomery et al. (1998) Proc Natl Acad Sci USA 95:15502-15507), virus-induced gene silencing (Burton et al. (2000) Plant Cell 12:691-705; and Baulcombe (1999) Curr Op Plant Bio 2:109-113); ribozymes (Steinecke et al. (1992) EMBO J. 11:1525; Haseloff et al. (1988) Nature 334: 585-591; U.S. Pat. No. 4,987,071; and, Perriman et al. (1993) Antisense Res Dev 3:253); oligonucleotide-mediated targeted modification (e.g., WO03/076574, and WO99/25853); Zn-finger targeted molecules (e.g., WO01/52620; WO03/048345; and WO00/42219); microRNA (miRNA) and/or siRNAs (e.g., US2005/0138689; and US2005/0120415); and any other methods, or any combinations thereof.

In other examples, the polynucleotide may be an artificial chromosome, including but not limited to a BAC or YAC clone or derivatives thereof, or any polynucleotide comprising a plant or a maize centromeric region. In some examples the polynucleotide comprises a minichromosome polynucleotide. A minichromosome polynucleotide includes satellite minichromosomes, artificial chromosomes, supernumerary chromosomes, chromosome fragments, and the like that are stably transmitted to a daughter cell during mitosis. In some examples, the polynucleotide comprises a mixture of minichromosome polynucleotides including but not limited to DNA constructs, genomic fragments, BAC or YAC clone(s) comprising a plant or maize centromeric repeat, genomic fragment(s) comprising a plant centromeric repeat, a telomere, and/or an origin of replication functional in a plant or any combination thereof.

The composition of interest may include one or more polynucleotides, for example, a mixture of polynucleotide species. Accordingly, the composition of interest may include at least one polynucleotide species, and may include mixtures of polynucleotide species wherein each polynucleotide species in the mixture each has at least one distinct characteristic as compared to any other member of the mixture such as size, sequence composition, strandedness, physical form, modified bases, and the like. The combinations include multiple copies of any one of the polynucleotides of interest, and may have any combination of up-regulating and down-regulating expression of the combined polynucleotides. The combinations may or may not be combined on one polynucleotide and may be provided sequentially or simultaneously.

The polynucleotide for delivery to the plant cell may encode a polypeptide of interest which is expressed in the cell. The polynucleotide may encode a bioactive protein or polypeptide. The polynucleotide delivered to the plant cell may confer a particular trait of interest to the plant, for example, such as, but not limited to an agronomic trait, a disease resistance trait, an insect resistance trait, a herbicide tolerance trait, modified nitrogen use, modified water use, drought tolerance, nutritional enhancements, sugar content, oil traits, starch traits, carbohydrate, nutrient metabolism, increased oil production, increased protein production, unique oil and protein production, increased fermentable starch production, increased amino acid content, increased fatty acids content, modified flower senescence, commercial processing traits, enhanced digestibility, male sterility, grain traits, or modified growth rate. Examples of agronomic traits include yield, yield stability, abiotic stress tolerance, drought tolerance, cold stress tolerance, heat stress tolerance, stalk qualities, root mass, canopy characteristics, lodging, nitrogen utilization, seed set, seed size, plant height, ear height, and the like. Herbicide resistance traits include resistance to herbicides that inhibit acetolactate synthase (ALS), such as sulfonylurea-type herbicides; resistance to herbicides that inhibit glutamine synthase, such as phosphinothricin or bialaphos, resistance to herbicides in the hydroxyphenyl pyruvate dioxygenase (HPPD)-inhibiting family, and resistance to glyphosate.

Insect resistance genes may encode resistance to pests such as rootworm, cutworm, armyworm, earworm, European Corn Borer, and the like. Such genes include Bacillus thuringiensis (Bt) toxic protein genes. Disease resistance traits include resistance or tolerance against to Fusarium spp., Phytopthera spp., Pseudomonas spp., Erwinia spp., Gibberella spp., Sclerotinia spp., anthracnose, white mold, stalk rots, root rots, ear molds, leaf spots, rust, blights, mildews, nematodes, and/or viral diseases.

Male sterility can be conferred by genes including male tissue-preferred genes and genes with male sterility phenotypes such as QM. Other genes include kinases and those encoding compounds toxic to either male or female gametophytes. In some examples male sterility is provided via RNAi molecules or polynucleotides encoding RNAi molecules.

Exemplary compositions of interest provided to a plant cell include polypeptides, and/or polynucleotides (DNA or RNA) encoding a screenable marker, a polypeptide that can enhance or stimulate cell growth, an enzyme such as a recombinase, an integrase, a site-specific recombinase, a homing endonuclease, a transposase, a meganuclease, a restriction enzyme, a transcription factor, a repressor, and/or a zinc-finger protein.

Any number of screenable markers may be delivered, for example, to facilitate the identification cells containing the marker or recovery of transformed cells comprising the marker. The marker(s) may be encoded by a polynucleotide having plant preferred codons, for example, maize preferred codons. Screenable markers include, e.g., visual and/or selection markers. Screenable markers may be used to detect gene expression in the plant cell for identification of transformed cells. Exemplary screenable markers include, for example, fluorescent markers, e.g., green fluorescent protein (GFP), yellow fluorescent protein (YFP), enhanced green fluorescent protein (EGFP), red fluorescent protein (RFP), blue fluorescent protein (BFP), cyan fluorescent protein (CFP) and the like, or a luminescent protein (e.g., a firefly luciferase protein) and the like. In further examples, the visual marker comprises beta-glucuronidase (GUS), GFPm, AmCyan, ZsYellow, or DsRed. See, Wenck et al. (2003) Plant Cell Rep 22:244-251.

Screenable markers also include selectable markers including any marker that confers resistance to a selective agent. Selection markers and their corresponding selective agents include, but are not limited to, herbicide resistance genes and herbicides; antibiotic resistance genes and antibiotics; and other chemical resistance genes with their corresponding chemical agents. Exemplary selectable markers include bacterial drug resistance genes including but not limited to, neomycin phosphotransferase II (nptII) which confers resistance to kanamycin, paromycin, neomycin, and G418, and hygromycin phosphotransferase (hph) which confers resistance to hygromycin B. See also, Bowen (1993) Markers for Plant Gene Transfer, Transgenic Plants, Vol. 1, Engineering and Utilization; Everett et al. (1987) Bio/Technology 5:1201-1204; Bidney et al. (1992) Plant Mol Biol 18:301-313; and WO97/05829. Resistance may also be conferred to herbicides from several groups, including amino acid synthesis inhibitors, photosynthesis inhibitors, lipid inhibitors, growth regulators, cell membrane disrupters, pigment inhibitors, seedling growth inhibitors, including but not limited to imidazolinones, sulfonylureas, triazolopyrimidines, glyphosate, sethoxydim, fenoxaprop, glufosinate, phosphinothricin, triazines, bromoxynil, and the like (see, e.g., Holt (1993) Ann Rev Plant Physiol Plant Mol Biol 44:203-229; and Miki et al. (2004) J Biotechnol 107:193-232). Selectable marker genes and proteins include the bar gene encoding phosphinothricin acetyl transferase (PAT) which confers resistance to glufosinate (Thompson et al. (1987) EMBO J. 6:2519-2523); glyphosate oxidoreductase (GOX), glyphosate N-acetyltransferase (GAT), and 5-enol pyruvylshikimate-3-phosphate synthase (EPSPS) which confer resistance to glyphosate (Barry et al. (1992) in Biosynthesis and Molecular Regulation of Amino Acids in Plants, B. K. Singh et al. (Eds) pp. 139-145; Kishore et al. (1992) Weed Tech 6:626-634; Castle (2004) Science 304:1151-1154; Zhou et al. (1995) Plant Cell Rep 15:159-163; WO97/04103; WO02/36782; and WO03/092360); dihydrofolate reductase (DHFR), which confers resistance to methotrexate (see, e.g., Dhir et al. (1994) Improvements of Cereal Quality by Genetic Engineering, R. J. Henry (ed), Plenum Press, New York; and Hauptmann et al. (1988) Plant Physiol 86:602-606); acetohydroxy acid synthase (AHAS or ALS) mutant sequences lead to resistance to imidiazolinones and/or sulfonylureas such as imazethapyr and/or chlorsulfuron (see, e.g., Zu et al. (2000) Nat Biotechnol 18:555-558; U.S. Pat. Nos. 6,444,875, and 6,660,910; Sathasivan et al. (1991) Plant Physiol 97:1044-1050; Ott et al. (1996) J Mol Biol 263:359-368; and Fang et al. (1992) Plant Mol Biol 18:1185-1187). In addition, chemical resistance genes further include tryptophan decarboxylase which confers resistance to 4-methyl tryptophan (4-mT) (Goodijn et al. (1993) Plant Mol Biol 22:907-912); bromoxynil nitrilase which confers resistance to bromoxynil; cyanamide hydratase (Cah), (see, e.g., Greiner et al. (1991) Proc Natl Acad Sci USA 88:4260-4264; and Weeks et al. (2000) Crop Sci 40:1749-1754) which converts cyanamide into urea, thereby conferring resistance to cyanamide. Any form or derivative of cyanamide can be used as a selection agent including, but not limited to, calcium cyanamide (Perlka® (SKW, Trotberg Germany) and hydrogen cyanamide (Dormex® (SKW)). See also, U.S. Pat. Nos. 6,096,947, and 6,268,547.

In some examples the composition of interest comprises a polynucleotide or polypeptide that stimulates cell growth. Use of these compositions of interest may provide a means for positive selection of recipient target cells, increased transformation efficiency, increased plastid transformation efficiency, increased gene targeting or combinations thereof. Genes that enhance or stimulate cell growth include genes involved in transcriptional regulation, homeotic gene regulation, stem cell maintenance and proliferation, cell cycle regulation, cell division, and/or cell differentiation such as WOX family genes including WUS homologues (Mayer et al. (1998) Cell 95:805-815; WO01/0023575; US2004/0166563); aintegumenta (ANT) (Klucher et al. (1996) Plant Cell 8:137-153; Elliott et al. (1996) Plant Cell 8:155-168; GenBank Accession Nos. U40256, U41339, Z47554); clavata (e.g., CLV1, CVL2, CLV3) (WO03/093450; Clark et al. (1997) Cell 89:575-585; Jeong et al. (1999) Plant Cell 11:1925-1934; Fletcher et al. (1999) Science 283:1911-1914); Clavata and Embryo Surround region genes (e.g., CLE) (Sharma et al. (2003) Plant Mol Biol 51:415-425; Hobe et al. (2003) Dev Genes Evol 213:371-381; Cock & McCormick (2001) Plant Physiol 126:939-942; Casamitjana-Martinez et al. (2003) Curr Biol 13:1435-1441); babyboom (e.g., BNM3, BBM, ODP1, ODP2) (WO00/75530; Boutileir et al. (2002) Plant Cell 14:1737-1749); Zwille (Lynn et al. (1999) Dev 126:469-481); leafy cotyledon (e.g., Lec1, Lec2) (Lotan et al. (1998) Cell 93:1195-1205; WO00/28058; Stone et al. (2001) Proc Natl Acad Sci USA 98:11806-11811; U.S. Pat. No. 6,492,577); Shoot Meristem-less (STM) (Long et al. (1996) Nature 379:66-69); ultrapetala (ULT) (Fletcher (2001) Dev 128:1323-1333); mitogen activated protein kinase (MAPK) (Jonak et al. (2002) Curr Opin Plant Biol 5:415); kinase associated protein phosphatase (KAPP) (Williams et al. (1997) Proc Natl Acad Sci USA 94:10467-10472; Trotochaud et al. (1999) Plant Cell 11:393-406); ROP GTPase (Wu et al. (2001) Plant Cell 13:2841-2856; Trotochaud et al. (1999) Plant Cell 11:393-406); fasciata (e.g. FAS1, FAS2) (Kaya et al. (2001) Cell 104:131-142); cell cycle genes (U.S. Pat. No. 6,518,487; WO99/61619; WO02/074909), Shepherd (SHD) (Ishiguro et al. (2002) EMBO J. 21:898-908); Poltergeist (Yu et al. (2000) Dev 127:1661-1670; Yu et al. (2003) Curr Biol 13:179-188); Pickle (PKL) (Ogas et al. (1999) Proc Natl Acad Sci USA 96:13839-13844); knox genes (e.g., KN1, KNAT1) (Jackson et al. (1994) Dev 120:405-413; Lincoln et al. (1994) Plant Cell 6:1859-1876; Venglat et al. (2002) Proc Natl Acad Sci USA 99:4730-4735); fertilization independent endosperm (FIE) (Ohad et al. (1999) Plant Cell 11:407-415), cell cycle regulators (e.g., cyclins, cyclin-dependent kinases (CDKs), and the like. In some examples, the polypeptide(s) that enhances or stimulates cell growth is a cell cycle regulator (e.g., cyclin, cyclin-dependent kinase), a wuschel polypeptide, a babyboom polypeptide, or any combination thereof.

In some examples, the particle mixture is used to delivery compositions of interest for plastid transformation. In some examples, the composition(s) of interest for plastid transformation are associated with the nanoparticles. Nanoparticles have been used to deliver plasmid DNA to a variety of animal cells. It has been found that when DNA coated nanoparticles are incubated with cells lacking a cell wall, the cells take up the particles and begin expressing any product encoded by the DNA. Transport of protein, directly or indirectly provided by the nanoparticle-microparticle mixture, to a subcellular compartment, such as the chloroplast, vacuole, peroxisome, nucleus, glyoxysome, cell wall, mitochondrion, or for secretion into the apoplast, can be accomplished by operably linking a signal sequence to the N-terminus and/or C-terminus of the protein of interest. For example, a nucleotide sequence encoding a signal sequence can be operably linked to the 5′ and/or 3′ region of a polynucleotide encoding the protein of interest. Signal targeting sequences at the 5′ and/or 3′ end of the structural gene may determine, during polypeptide synthesis and processing, where the final encoded protein product may ultimately by compartmentalized. Alternatively, such subcellular compartment targeting proteins can be directly associated with a nanoparticle of the mixture to direct the associated nanoparticle to the desired subcellular compartment. The presence of a signal sequence directs a polypeptide to either an intracellular organelle, a subcellular compartment, or for secretion to the apoplast. Many signal sequences are known, including but not limited to those disclosed in Becker et al. (1992) Plant Mol Biol 20:49; Close (1993) Master's Thesis, Iowa State University; Knox et al. (1987) Plant Mol Biol 9:3-17; Lerner et al. (1989) Plant Physiol 91:124-129; Fontes et al. (1991) Plant Cell 3:483-496; Matsuoka et al. (1991) Proc Natl Acad Sci 88:834; Gould et al. (1989) J Cell Biol 108:1657; Creissen et al. (1991) Plant J; Kalderon et al. (1984) Cell 39:499-509; and, Steifel et al. (1999) Plant Cell 2:785-793. In some examples, the composition(s) of interest further comprise compositions for enhancing cell growth, enhancing gene targeting, and/or for stable transformation of the nuclear genome. In some examples, the composition(s) of interest further comprise a wuschel, a babyboom, a cell cycle regulator, a hormone, a hormone antagonist, a recombinase, a transfer cassette, a homing endonuclease, a ligand, a marker, or any combinations thereof.

In some examples the composition of interest may comprise compounds for gene targeting. Gene targeting includes any sequence modification at a specific selected target locus, including but not limited to nucleotide substitutions, deletions, and insertions, and polynucleotide excisions, insertions, and replacements. A target locus is a specific sequence and/or location in a genome (e.g., nuclear, chloroplast, mitochondrial, plastid). A target site comprises at least one recognition sequence. A recognition sequence or recognition site refers to any nucleotide sequence that is specifically recognized and/or bound by a double-strand break-inducing enzyme. In some examples, a transfer or donor cassette is provided. A transfer cassette comprises a polynucleotide of interest that directs a modification at the target locus. For example, the transfer cassette may comprise a polynucleotide of interest to be inserted at the target site. In some examples, the transfer cassette may comprise a recognition site. Examples of double-strand break inducing enzymes include site-specific recombinases, restriction enzymes, homing endonucleases. In some examples, a double-strand break inducing enzyme has been designed and modified to recognize an endogenous genomic target site. Gene targeting includes any site-specific system such as site-specific recombinase systems, and homologous recombination systems.

A double-strand break-inducing enzyme is any enzyme that recognizes and/or

binds to a specific recognition sequence to produce a double-strand break at or near the recognition sequence. The double-strand break could be the direct action of the enzyme itself, or the enzyme might introduce a single-stranded nick in the DNA that then leads to a double-strand break induced by other cellular machinery (e.g., cellular repair mechanisms). Examples of double-strand break-inducing enzymes include, but are not limited to, endonucleases, site-specific recombinases, transposases, topoisomerases, and zinc finger nucleases, and include modified derivatives, variants, and fragments thereof. A modified double-strand break-inducing enzyme can be derived from a native, naturally-occurring double-strand break-inducing enzyme, or it can be artificially created or synthesized. Those modified double-strand break-inducing enzymes that are derived from a native, naturally-occurring double-strand break-inducing enzymes can be modified to recognize a different recognition sequence (at least one nucleotide difference) than its native form. In certain examples, the double-strand break inducing enzyme recognition sequences are of a sufficient length to have only one copy in a genome of an organism.

In some examples, the double-strand break-inducing enzyme is an endonuclease. Endonucleases are enzymes that cleave the phosphodiester bond in a polynucleotide, and include restriction endonucleases that cleave DNA at specific sites without damaging the bases. Restriction endonucleases include Type I, Type II, Type III, and Type IV endonucleases, which further include various subtypes. In the Type I and Type III systems, a single protein complex has both methylase and restriction activities. Type IV restriction enzymes target methylated DNA. Restriction enzymes are further described and classified, for example in the REBASE database (on the world wide web at rebase.neb.com).

Endonucleases useful in the methods and compositions include homing endonucleases, which bind and cut polynucleotides at a specific recognition sequence that are typically longer than those of restriction enzymes, about 18 bp or more. These

sequences are predicted to naturally occur infrequently in a genome, typically only one or
two sites per genome. Homing endonucleases (also called meganucleases) have been classified into four families based on conserved sequence motifs: the LAGLIDADG, GIY-YIG, H—N—H, and His-Cys box families. These motifs participate in the coordination of metal ions and hydrolysis of phosphodiester bonds. Homing endonucleases are notable for their long recognition sites, and for tolerating some sequence polymorphisms in their DNA substrates. The naming convention for homing endonucleases is similar to the convention for other restriction endonucleases. Homing endonucleases are also characterized by a prefix of F-, I-, or PI- for enzymes encoded by free-standing ORFs, introns, and inteins, respectively. For example, the intron-, intein-, and freestanding gene-encoded homing endonucleases from Saccharomyces cerevisiae are denoted I-SceI, PI-SceI, and F-SceII (HO endonuclease), respectively. Homing endonuclease domains, structure and function are known (see e.g., Guhan & Muniyappa (2003) Crit. Rev Biochem Mol Biol 38:199-248; Lucas et al. (2001) Nucleic Acids Res 29:960-9; Jurica & Stoddard (1999) Cell Mol Life 5 Sci 55:1304-26; Stoddard (2006) Q Rev Biophys 38:49-95; and Moure et al. (2002) Nat Struct Biol 9:764, each of which is herein incorporated by reference). In some examples, a naturally occurring variant, and/or an engineered derivative homing endonuclease is used. The cleavage specificity of a homing endonuclease can be changed by rational design of amino acid substitutions at the DNA binding domain and/or combinatorial assembly and selection of mutated monomers (see, for example, Arnould et al. (2006) J Mol Biol 355:443-58; Ashworth et al. (2006) Nature 441:656-9; Doyon et al. (2006) J Am Chem Soc 128:2477-84; Rosen et al. (2006) Nucleic Acids Res 34:4791-800; and Smith et al. (2006) Nucleic Acids Res 34:e149, each of which is herein incorporated by reference). Engineered homing endonucleases have been demonstrated that can cleave cognate mutant sites without broadening their specificity. The endonuclease includes a modified endonuclease that binds a non-native or heterologous recognition sequence, and does not bind a native or endogenous recognition sequence. An engineered or modified endonuclease can have only a single modified amino acid or many amino acid changes. Methods for modifying the kinetics, cofactor interactions, expression, optimal conditions, and/or recognition site specificity of homing endonucleases, and subsequently screening for activity are known, see e.g., Epinat et al. (2003) Nucleic Acids Res 31:2952-62; Chevalier et al. (2002) Mol Cell 10:895-905; Gimble et al. (2003) Mol Biol 334:993-1008; Seligman et al. (2002) Nucleic Acids Res 30:3870-9; Sussman et al. (2004) J Mol Biol 342:31-41; Rosen et al. (2006) Nucleic Acids Res 34:4791-800; Chames et al. (2005) Nucleic Acids Res 33:e178; Smith et al. (2006) Nucleic Acids Res 34:e149; Gruen et al. (2002) Nucleic Acids Res 30:e29; Chen & Zhao, (2005) Nucleic Acids Res 33:e154; US2007/0117128; WO05/105989, WO03/078619, WO06/097854, WO06/097853, WO06/097784, WO04/031346, WO04/067753, and WO07/047,859, each of which is herein incorporated by reference in its entirety.

Any homing endonuclease can be used as a double-strand break inducing agent

including, but not limited to, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsblVP, I-TIiI, IPpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, 5 I-DmoI, I-HmuI, IHmuII, I-HsNIP, I-LIaI, I-MsoI, I-NaaI, I-NanI, I-NcIIP, I-NgrIP, I-NitI, I-NjaI, INsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-Scat, I-SexIP, I-SneIP, I-SpomI, I-SpomCP, I-SpomIP, ISpomIIP, I-SquIP, I-Ssp68031, I-SthPhiJP, I-SthPhiST3P, I-SthPhiSTe3bP, I-TdeIP, I-Tev-I, I-TevII, I-TevIII, I-UarAP, I-UarHGPAIP, I-UarHGPA13P, I-VinIP, I-ZbiIP, PIMtuI, PI-MtuHIP PI-MtuHIIP, PI-PfuI, PI-PfuII, PI-PkoI, PI-PkoII, PI-Rma43812IP, PISpBetaIP, PI-SceI, PI-TfuI, PI-TfuII, PI-ThyI, PI-TIiI, PI-TIiII, or any variant or derivative thereof.

In other examples, the double-strand break-inducing enzyme is a zinc finger nuclease. Zinc finger nucleases (ZFNs) are engineered double-strand break inducing agents comprised of a zinc finger DNA binding domain and a double strand break-inducing enzymatic domain. Recognition site specificity is conferred by the zinc finger domain, which typically comprises two, three, four, or more zinc fingers, for example having a C2H2 structure; however other zinc finger structures are known and have been engineered. Zinc finger domains are amenable to the design of polypeptides which specifically bind a selected polynucleotide recognition sequence. Some ZFNs consist of an engineered DNA-binding zinc finger domain linked to a non-specific endonuclease domain, for example, a nuclease domain from a Type IIs endonuclease such as FokI. Additional functionalities can be fused to the zinc-finger binding domain, including transcriptional activator domains, transcription repressor domains, and methylases. In some examples, dimerization of the nuclease domain is required for cleavage activity. Each zinc finger recognizes three consecutive base pairs in the target DNA. For example, a 3-finger domain recognizes a sequence of nine contiguous nucleotides, with a dimerization requirement of the nuclease. A recognition sequence of 18 nucleotides is long enough to be unique in a genome (4¹⁸=6.9×10¹⁰).

Zinc finger modules predominantly recognize GNN and ANN triplets (Dreier et al. (2001) J Biol Chem 276:29466-78; Dreier et al. (2000) J Mol Biol 303:489-502; Liu et al. (2002) J Biol Chem 277:3850-6), but examples using CNN or TNN triplets are also known

### (Dreier et al. (2005) J Biol Chem 280:35588-5 97; Jamieson et al. (2003) Nature Rev Drug

Discov 2:361-8). See also, Durai et al. (2005) Nucleic Acids Res 33:5978-90; Segal (2002) Methods 26:76-83; Porteus & Carroll (2005) Nat Biotechnol 23:967-73; Pabo et al. (2001) Ann Rev Biochem 70:313-40; Wolfe et al. (2000) Ann Rev Biophys Biomol Struct 29:183-212; Segal & Barbas (2001) Curr Opin Biotechnol 12:632-7; Segal et al. (2003) Biochemistry 42:2137-48; Beerli & Barbas (2002) Nat Biotechnol 20:135-41; Mani et al. (2005) Biochem Biophys Res Comm 335:447-57; Lloyd et al. (2005) Proc Natl Acad Sci USA 102:2232-7; Carroll et al. (2006) Nature Protocols 1:1329; Ordiz et al. (2002) Proc Natl Acad Sci 99:13290-5; Guan et al. (2002) Proc Natl Acad Sci 99:13296-301; Townsend et al. (2009) Nature 459:442-445; Sander et al. (2008) Nucl Acids Res 37:509-515; Fu et al. (2009) Nucl Acids Res 37:D297-283; Maeder et al. (2008) Mol Cell 31:294-301; Wright et al. (2005) Plant J 44:693-705; Wright et al. (2006) Nat Prot 1:1637-1652; zinc-finger consortium (website at www-dot-zincfingerdot-org); WO02/099084; WO00/42219; WO02/42459; WO03/062455; US2003/0059767, US2003/0108880; and U.S. Pat. Nos. 6,534,261, 7,262,054, 7,378,510, 7,151,201, 6,140,466, 6,511,808 and 6,453,242; each of which is herein incorporated by reference in its entirety.

Elements from recombination systems, such as recombinases, and recombination sites can be provided, for example as a polypeptide, as an RNA, as a DNA, in a DNA construct, a target site, a transfer cassette, or any combination thereof. In some examples, a target site comprises at least one site-specific recombinase recognition site. Optionally, the target site polynucleotide comprises a promoter operably linked to at least one recombination site. Any promoter can be used, and is typically selected based on the desired outcome.

Any site-specific recombination system or component of thereof may be used. A site-specific recombinase, is a polypeptide that catalyzes conservative site-specific recombination between its compatible recombinogenic recombination sites, and includes wild type sequences as well as a wide variety of modified sites that retain activity. A site-specific recombination site, or recombination site, is a polynucleotide sequence recognized by a site-specific recombinase as a substrate for the site-specific recombination reaction, and includes wild type sequences as well as a wide variety of modified sites that retain activity. For reviews of site-specific recombinases, see Sauer (1994) Curr Op Biotechnol 5:521-527; and Sadowski (1993) FASEB 7:760-767. Recombinases include recombinases from the integrase and the resolvase families. The integrase family of recombinases has over one hundred members and includes, for example, FLP, Cre, lambda integrase, and R. For other members of the integrase family, see for example, Esposito et al. (1997) Nucleic Acids Res 25:3605-3614 and Abremski et al. (1992) Protein Eng 5:87-91. Other recombination systems include, for example, the streptomycete bacteriophage phi C31 (Kuhstoss et al. (1991) J Mol Biol 20:897-908); the SSV1 site-specific recombination system from Sulfolobus shibatae (Maskhelishvili et al. (1993) Mol Gen Genet. 237:334-342); and a retroviral integrase-based integration system (Tanaka et al. (1998) Gene 17:67-76).

FLP recombinase catalyzes a site-specific reaction that is involved in amplifying the copy number of the two-micron plasmid of S. cerevisiae during DNA replication. FLP recombinase catalyzes site-specific recombination between two FRT sites. The FLP protein has been cloned and expressed (Cox (1993) Proc Natl Acad Sci USA 80:4223-4227). The FLP recombinase may be derived from the genus Saccharomyces. One can also synthesize a polynucleotide comprising the recombinase using plant-preferred codons for enhanced expression in a plant of interest (e.g., U.S. Pat. No. 5,929,301). Additional functional variants and fragments of FLP are known (Buchholz et al. (1998) Nat Biotechnol 16:617-618, Hartung et al. (1998) J Biol Chem 273:22884-22891, Saxena et al. (1997) Biochim Biophys Acta 1340:187-204, and Hartley et al. (1980) Nature 286:860-864). FRT variants include minimal sites (see, e.g., Broach et al. (1982) Cell 29:227-234; Senecoff et al. (1985) Proc Natl Acad Sci 82:7270-7274; Gronostajski & Sadowski (1985) J Biol Chem 260:12320-12327; Senecoff et al. (1988) J Mol Biol 201:405-421; and WO99/25821), and variants (see, e.g., Schlake & Bode (1994) Biochemistry 33:12746-12751; Seibler & Bode (1997) Biochemistry 36:1740-1747; Umlauf & Cox (1988) EMBO J. 7:1845-1852; Senecoff et al. (1988) J Mol Biol 201:405-421; Voziyanov et al. (2002) Nucleic Acids Res 30:7; WO07/011,733, WO99/25854, WO99/25840, WO99/25855, WO99/25853 and WO99/25821; and U.S. Pat. Nos. 7,060,499 and 7,476,539; each of

which are herein incorporated by reference in its entirety). Non-limiting examples of recombination sites include FRT sites including, for example, the native FRT site (FRT1), and various functional variants of FRT, including but not limited to, FRT5, FRT6, FRT7, FRT12, and FRT87 (see, e.g., WO03/054189, WO02/00900, WO01/23545; and Schlake et al. (1994) Biochemistry 33:12745-12751, each of which is herein incorporated by reference).

The bacteriophage recombinase Cre catalyzes site-specific recombination between two lox sites. The Cre recombinase is known (Guo et al. (1997) Nature 389:40-46; Abremski et al. (1984) J Biol Chem 259:1509-1514; Chen et al. (1996) Somat Cell Mol Genet. 22:477-488; Shaikh et al. (1977) J Biol Chem 272:5695-5702; and, Buchholz et al. (1998) Nat Biotechnol 16:617-618. Cre polynucleotide sequences may also be synthesized using plant-preferred codons (e.g., moCre, WO99/25840). A chimeric recombinase can be also used, for example as described in WO99/25840. Cre recombinase catalyzes a site-specific recombination reaction by interacting with lox recombination sites. Besides the wild type lox site (loxP), many active lox site variants are well known and available. An analysis of the recombination activity of variant LOX sites is presented in Lee et al. (1998) Gene 216:55-65 and in U.S. Pat. No. 6,465,254. Also, see for example, Huang et al. (1991) Nucleic Acids Res 19:443-448; Sadowski (1995) In Progress in Nucleic Acid Research and Molecular Biology Vol. 51, pp. 53-91; Cox (1989) In Mobile DNA, Berg and Howe (eds) American Society of Microbiology, Washington D.C., pp. 116-670; Dixon et al. (1995) Mol Microbiol 18:449-458; Buchholz et al. (1996) Nucleic Acids Res 24:3118-3119; Kilby et al. (1993) Trends Genet. 9:413-421; Rossant & Geagy (1995) Nat Med 1:592-594; Albert et al. (1995) Plant J 7:649-659; Bayley et al. (1992) Plant Mol Biol 18:353-361; Odell et al. (1990) Mol Gen Genet. 223:369-378; Dale & Ow (1991) Proc Natl Acad Sci USA 88:10558-10562; Qui et al. (1994) Proc Natl Acad Sci 91:1706-1710; Stuurman et al. (1996) Plant Mol Biol 32:901-913; Dale et al. (1990) Gene 91:79-85; and WO01/111058; each of which is herein incorporated by reference in its entirety.

The recombination sites employed in the methods and compositions can be identical sequences or dissimilar sequences. Recombination sites with dissimilar sequences can be either recombinogenic or non-recombinogenic with respect to one another. Recombinogenic describes a set of recombination sites (i.e., dissimilar or identical) that are capable of recombining with each another. Non-recombinogenic describes a set of recombination sites, in the presence of the appropriate recombinase, that will not recombine with one another or recombination between the sites is minimal. Accordingly, it is recognized that any suitable set of nonrecombinogenic and/or recombinogenic recombination sites may be utilized, including a FRT site or functional variant thereof, a LOX site or functional variant thereof, any combination thereof, or any other combination of non-recombinogenic and/or recombination sites known. The orientation of any two sites relative to each other will determine the recombination reaction product. In some examples, the recombination sites are asymmetric. Directly repeated recombination sites are arranged in the same orientation, such that recombination between these sites results in excision, rather than inversion, of the intervening DNA sequence. Inverted recombination sites are those recombination sites in a set of recombinogenic recombination sites that are arranged in the opposite orientation, so that recombination between these sites results in inversion, rather than excision, of the intervening DNA sequence.

In some examples the particle mixture comprises a site-specific recombinase polypeptide or a polynucleotide encoding the site-specific recombinase. For example, the target plant cell may comprise a target site comprising a polynucleotide of interest flanked by recombinogenic recombinase recognition sites. The mixture of particles is used to deliver the recombinase to the target plant cell, whereby the recombinase catalyzes excision of the polynucleotide of interest. When a recombinase polypeptide or recombinase mRNA is delivered, the recombinase activity is transient, and no recombinase polynucleotide can be incorporated into a genome. If a DNA construct encoding a recombinase is delivered, the DNA may be designed, modified, and/or associated with the particle mixture in such a way to reduce integration of the construct into a genome.

In some examples, a transfer or donor cassette is provided. In site-specific recombination systems, a transfer cassette comprises a polynucleotide of interest flanked by at least one recombination site which is recombinogenic with a recombination site at the target site in the host cell. If the transfer cassette is on a circular polynucleotide, one recombination site is sufficient for integration at the target site. In some examples, the desired end-product is a recombinase-mediated cassette exchange, wherein a target site comprising two non-recombinogenic recombination sites is reacted with a transfer cassette comprising the polynucleotide of interest flanked by two recombination sites identical to the sites at the target site such that in the presence of the recombinase, the sequence between the recombination sites at the target is replaced by the polynucleotide of interest from the transfer cassette. In some examples a transfer cassette comprises at least a first recombination site operably linked to a polynucleotide of interest and/or a polynucleotide encoding a screenable marker, where the first recombination site is recombinogenic with a recombination site in the target site. A targeted seed or plant has stably incorporated into its genome a DNA construct that has been generated and/or manipulated through the use of a recombination system. Site-specific recombination methods that result in various integration, alteration, and/or excision events to generate the recited DNA construct can be employed to generate a targeted seed. See, e.g., WO99/25821, WO99/25854, WO99/25840, WO99/25855, WO99/25853, WO99/23202, WO99/55851, WO01/07572, WO02/08409, and WO03/08045.

The composition of interest to be delivered may include one or more polypeptides. The polypeptide may be of any length, form and composition suitable to achieve the desired result. Accordingly, the polypeptide may be a peptide, protein, oligopeptide, dimer, multimer, and the like and may include a full-length polypeptide or a fragment thereof. Polypeptides are any amino acid polymer and include any naturally occurring, synthetic, or modified amino acids or a combination thereof. Polypeptide also includes those that are modified, such as amino acid deletions, additions and substitutions. The polypeptide may be in any suitable physical confirmation including linear, circular, branched, secondary, tertiary, and quaternary structures, and any combination thereof.

In some instances, the composition of interest comprises one or more polypeptides, for example, a mixture of polypeptide species. The composition of interest may include at least one polypeptide species and may include a mixture of polypeptide species where each polypeptide species in the mixture each has at least one distinct characteristic as compared to any other member of the mixture such as size, sequence composition, physical form, modified amino acid, and the like. In some examples the composition of interest comprises at least one polypeptide that can enhance or stimulate cell growth, an enzyme such as a recombinase, an integrase, a site-specific recombinase, a homing endonuclease, a transposase, a meganuclease, a restriction enzyme, a DNA binding protein, including but not limited to a DNA repair protein, a transactivating factor, leucine-zipper protein, a zinc-finger protein, a cell cycle protein, a DNA polymerase, a DNA ligase, and the like, a RNA binding protein, including but not limited to a DICER, a DICER-LIKE protein, a Drosha, a Rnase, a RNA-dependent RNA polymerase, ribosomal proteins, a transcription factor, a repressor, a screenable marker and the like. In some examples the polypeptide that enhances or stimulates cell growth includes without limitation a wuschel polypeptide or a babyboom polypeptide. In one example, the composition of interest includes a microorganism.

Examplary microorganisms include a bacterium such as Agrobacterium, a virus, a fungus, or a combination thereof. A variety of bacterial strains may be introduced into a plant. An Agrobacterium comprising a T-DNA containing a polynucleotide of interest is provided to a plant cell by direct delivery via particles, wherein the Agrobacterium is capable of T-DNA transfer into a plant cell. A number of wild-type and disarmed strains of Agrobacterium tumefaciens and Agrobacterium rhizogenes harboring T-DNA, Ti or Ri plasmids can be used. Agrobacterium can be provided directly to cells by particle bombardment as described in U.S. Pat. No. 5,932,782, or co-incubation with bombardment-wounded cells as described in EP 0486233, both of which are herein incorporated by reference. Agrobacterium strains of interest can be wild type or derivatives thereof which have alterations that increase transformation efficiency. Strains of interest include, but are not limited to, A. tumefaciens strain C58, a nopaline-type strain (Deblaere et al. (1985) Nucleic Acids Res 13:4777-4788); octopine-type strains such as LBA4404 (Hoekema et al. (1983) Nature 303:179-180); or succinamopine-type strains e.g., EHA101 or EHA105 (Hood et al. (1986) J Bacteriol 168:1291-1301); A. tumefaciens strain A281 (US2002/0178463); GV2260 (McBride et al. (1990) Plant Mol Biol 14:269-276); GV3100 and GV3101 (Holsters et al. (1980) Plasmid 3:212-230); A136 (Watson et al. (1975) J Bacteriol 123:255-264); GV3850 (Zambryski et al. (1983) EMBO J. 2:2143-2150); GV3101::Pmp90 (Koncz et al. (1986) Mol Gen Genet. 204:383-396); and, AGL-1 (Lazo et al. (1991) Biotechnology 9:963-967). Transfer DNA or T-DNA comprises a genetic element that is capable of integrating a polynucleotide contained within its borders into another polynucleotide. The T-DNA can comprise the entire T-DNA, but need only comprise the minimal sequence necessary for cis transfer, typically the right or left border is sufficient. The T-DNA can be synthetically derived or can be from an A. rhizogene Ri plasmid or from an A. tumefaciens Ti plasmid, or functional derivatives thereof. Any polynucleotide to be transferred, may be positioned adjacent to a single border sequence, or between a left border sequence and/or a right border sequence of the T-DNA. The sequences of the left and right border sequences may or may not be identical and may or may not be inverted repeats of one another. It is also possible to use only one border, or more than two borders, to accomplish transfer of a desired polynucleotide. Various plasmids are available comprising T-DNAs that can be employed in the methods. Examples of superbinary vectors include pTOK162 and pTIBo542 (US2002/178463 and JP Application No. 4-222527); pTOK23 (Komari et al. (1990) Plant Cell Rep 9:303-306); pPHP10525 (U.S. Pat. No. 6,822,144), see, also Ishida et al. (1996) Nat Biotech 14:745-750. Additional transformation vectors comprising T-DNAs that can be used further include, but are not limited to, pBIN19 (Bevan et al. (1984) Nucleic Acids Res 12:8711-8721); pC22 (Simoens et al. (1986) Nucleic Acids Res 14:8073-8090); pGA482 (An et al. (1985) EMBO J. 4:277-284); pPCV001 (Koncz et al. (1986) Mol Gen Genet. 204:383-396); pCGN1547 (McBride et al. (1990) Plant Mol Biol 14:269-276); pJJ1881 (Jones et al. (1992) Transgenic Res 1:285-297); pPzP111 (Hajukiewicz et al. (1994) Plant Mol Biol 25:989-994); and, pGreen0029 (Hellens et al. (2000) Plant Mol Biol 42:819-832).

In one example, the bacterium is an Agrobacterium delivered to a plant cell. For example, an Agrobacterium comprising a T-DNA containing a polynucleotide of interest may be associated with the particle mixture. The microorganism associated with the microparticle or nanoparticle may have an improved viability as compared to another method, for example, a control method that includes drying an Agrobacterium cell suspension in growth media onto the microparticles. The mixture of particles can be used to deliver the Agrobacterium and its T-DNA to a plant cell thereby transferring the polynucleotide of interest to the plant cell. The polynucleotide of interest may be stably incorporated into a genome of a plant cell. Optionally, the particle mixture may further comprise additional compositions of interest that may enhance or facilitate the transfer or integration of the polynucleotide of interest. In one example, the particle mixture may include acetosyringone to enhance the response to Agrobacterium. In some examples, acetosyringone may be chemically associated or linked to a nanoparticle and/or a microparticle of the mixture. In some examples the acetosyringone may be loaded into pores of the nanoparticle, which may optionally be capped. In other examples, the particle mixture may further comprise at least one vir gene or vir polypeptide. The vir polynucleotide(s) and/or vir polypeptide(s) may be chemically associated or linked to a nanoparticle and/or a microparticle of the mixture. In some examples the vir polynucleotide(s) and/or vir polypeptide(s) may be loaded into pores of the nanoparticle, which may optionally be capped.

The composition of interest to be delivered may comprise a subcellular organelle composition. In some examples the subcellular organelle is a plastid, such as a chloroplast or a mitochondrion, or a nucleus for example to provide a means for in vitro fertilization of a eukaryotic cell.

In some examples, one or more compositions of interest are delivered using the mixture of particles. The compositions may be located in the same or different regions of the nanoparticle or microparticle or both. In some cases, the compositions of interest for delivery to a plant cell are a polynucleotide and a polypeptide. As will be understood by one skilled in the art, depending on the specific sequences of the polynucleotide and a polypeptide, the composition may be delivered as a complex, e.g. a protein-polynucleotide complex such as a DNA or RNA binding protein and the appropriate cognate polynucleotide. Exemplary DNA and RNA binding proteins are known. In other instances, the polynucleotide and the polypeptide are not delivered in the form of a complex but the complex is formed subsequent to delivery to the plant cell where they are released.

The association of the composition of interest to the surface of the nanoparticle or microparticle may occur via any bonding or interaction mechanism, including, but not limited to, ionic bonding, hydrogen bonding, covalent bonding, non-covalent bonding such as Van der Waals bonding, metal bonding such as metal aromoticity and bonding through hydrophilic/hydrophobic interactions. Those of ordinary skill in the art may readily select the appropriate attachment technique for the type of composition of interest. In some examples the composition of interest is attached to the surface of the nanoparticle or microparticle using a lipid compound. Any compound that will attach or associate the composition of interest to the nanoparticle or the microparticle can be used. In some examples, the particles are individually or collectively associated with the composition of interest in the presence of Tfx-10™, Tfx-20™, Tfx-50™, Lipofectin™, Lipofectamine™, Cellfectin™, Effectene™, Cytofectin GSV™, Perfect Lipids™ DOTAP, DMRIE-C, FuGENE-6™, Superfect™, Polyfect™, polyethyleneimine (PEI), chitosan, protamine CI, DNA binding proteins, histone H1, histone CENH3, poly-L lysine, DMSA, and the like. In some examples the compound is a cationic lipid solution comprising N,N,N′,N′-tetramethyl-N,N′-bis(2-hydroxylethyl)-2,3-di(oleoyloxy)-1,4-butanediammonium iodide. In some examples the cationic lipid solution further comprises L-dioleoyl phosphatidylethanolamine (DOPE). When the composition of interest to be delivered is a polynucleotide, using the methods and compositions described the polynucleotide may be more uniformly deposited on the particle as compared to a standard control, for example, a CaCl₂-spermidine precipitation of DNA onto the microparticle. Further, the particles having the polynucleotide associated thereto by means of a lipid compound or cationic compound results in a more uniform suspension as compared to a control, for example, a CaCl₂-spermidine precipitation standard control. Using these methods, the frequency of delivery of a polynucleotide as a composition of interest is increased as compared to a control, for example, a CaCl₂-spermidine precipitation standard control.

The loading of one or more regions of the nanoparticles or surface of the microparticles or a combination thereof provides for multiple options for delivering various compositions. In some instances it may be desirable to utilize the mixture of particles to deliver one or more compositions of interest to the plant cell. Use of the methods and compositions described herein also provides for the opportunity to control the release of the compositions in a predetermined manner, for example, with respect to delayed-release, timed-release or sequential release of compositions from the nanoparticle-microparticle mixture. The length of the delay between the release of the compositions may depend on the location of the composition of interest in relation to the nanoparticle or microparticle. In one example, the composition may be released at an appropriate time by providing the appropriate conditions for its release as described elsewhere herein. Accordingly, the release of the one or more compositions may be independently releasable or dependent on a condition being met to trigger its release.

In some examples, the compositions of interest may be released simultaneously or sequentially, for example, in a time-delayed manner. To achieve the desired release profile, the nanoparticle may be formed of certain materials and certain techniques used so the morphology of the nanoparticles with the desired properties are created, e.g. multi-layered structure and multi-pores. The release of the composition from the nanoparticle-microparticle mixture may be due to diffusion, due to degradation of the core, the layer, or pore, and/or breaking or cleavage of a bond which attaches the composition to the nanoparticle or microparticle, removal of a cap, or by any other suitable release mechanism. For example, if a nanoparticle is formed so that it has polynucleotides attached to its exterior surface, a protein incorporated within its layers, and pores containing a hormone that are capped with a nanoparticle then it is conceivable that the polynucleotides would be released first, the proteins released next when the layers degrade and the hormone released when the cap is removed. The ability to control the release of the compositions in a predetermined manner allows for the manipulation of gene expression in a plant cell. In some examples a polynucleotide encoding a protein of interest driven by a chemically induced promoter is attached to the microparticle and the appropriate chemical inducer is loaded into the pores of the nanoparticle and capped. The pores are subsequently uncapped when the nanoparticle reaches the target site of the plant cell and the chemical inducer released at an appropriate time so that expression of the chemically induced gene occurs.

Gene expression in the plant cell may be further manipulated by the duration of expression. When the composition of interest is a polynucleotide, the released polynucleotide may be stably incorporated into a genome of the plant cell to produce a transformed cell. As appreciated by one skilled in the art, the presence of some released compositions of interest such as a drug, hormone, polypeptide or polynucleotide delivered to the plant cell may be transient in nature. Accordingly, upon delivery into the plant cell, at one or more of the compositions of interest may dissociate from the microparticle or nanoparticle or both. Other compositions of interest such as a polynucleotide may stably integrate into the genome of the plant cell to produce a transformed cell, while yet still other compositions of interest may remain associated with the nanoparticle or microparticle or both, for example providing a means for transient delivery or expression of a polynucleotide or polypeptide of interest. In one aspect, the association agent used comprises PEI.

In general, the mixtures of particles comprising nanoparticles attached to microparticles may be delivered to plant cells according to known techniques that utilize biolistic-mediated transformation also commonly referred to as particle bombardment, biolistics, microprojectile bombardment, particle acceleration, particle inflow gun, or gene gun. Generally the target plant cells are competent for transformation and regeneration.

Cells and/or tissue from any plant species can be used with the methods and compositions, including, but not limited to, plant cells and/or tissue from a monocotyledonous or a dicotyledonous plant. In some examples the plant cell and/or tissue is selected from the group consisting of maize, rice, wheat, barley, millet, sorghum, rye, sugarcane, soybean, alfalfa, canola, Arabidopsis, tobacco, sunflower, cotton, and safflower. Examples of plant genera and species include, but are not limited to, maize (Zea mays), Brassica sp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), legumes including guar beans, locust bean, fenugreek, garden beans, cowpea, mungbean, fava bean, lentils, and chickpea, vegetables, ornamentals, grasses and conifers. Vegetables include tomatoes (Lycopersicon esculentum), lettuce (e.g., Lactuca sativa), green beans (Phaseolus vulgaris), lima beans (Phaseolus limensis), peas (Pisium spp., Lathyrus spp.), and Cucumis species such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers include pines, for example, loblolly pine (Pinus taeda), slash pine (Pinus effiotii), ponderosa pine (Pinus ponderosa), lodgepole pine (Pinus contorta), and Monterey pine (Pinus radiata), Douglas fir (Pseudotsuga menziesii); Western hemlock (Tsuga canadensis), Sitka spruce (Picea glauca), redwood (Sequoia sempervirens), true firs such as silver fir (Abies amabilis) and balsam fir (Abies balsamea), and cedars such as Western red cedar (Thuja plicata) and Alaska yellow cedar (Chamaecyparis nootkatensis).

Plant cells, plants, and seeds produced by the method and comprising the composition of interest are provided. The plant cells can be in any suitable form. For example, the plant cells can be included within an intact plant, explant, and/or seed. The term plant includes plant cells, protoplasts, plant cell tissue cultures, explants, and/or suspension cultures, plant calli, plant clumps, and plant cells that are intact in plants or parts of plants such as embryos, meristems, pollen, ovules, seeds, pods, stems, leaves, flowers, branches, fruit, kernels, ears, cobs, husks, stalks, roots, root tips, anthers, and the like. In some examples, a plant can be regenerated from the plant cells. Progeny, variants, and mutants of the regenerated plants are also included. Plant cells, plant callus, explants, organs, or parts thereof can be regenerated to form plants. Regeneration techniques are described generally in Klee et al. Ann Rev Plant Phys (1987) 38:467-486. The cells produced by the methods may be grown into plants using standard techniques and media (e.g., McCormick et al. (1986) Plant Cell Rep 5:81-84; Gruber et al. (1993) Vectors for Plant Transformation, In: Methods in Plant Molecular Biology and Biotechnology; Glick & Thompson, Eds., CRC Press, Inc., Boca Raton, pages 89-119; Gordon-Kamm et al. (1990) Plant Cell 2:603-618). These plants may then be grown and self-pollinated, backcrossed, and/or outcrossed, and the resulting progeny having the desired characteristic(s) identified. Two or more generations may be grown to ensure that the characteristic is stably maintained and inherited and then seeds harvested. In this manner transformed/transgenic seed having a recited a construct stably incorporated into their genome are provided. A plant and/or a seed having stably incorporated the construct can be further characterized for expression, site-specific integration potential, agronomics, and copy number (see, e.g., U.S. Pat. No. 6,187,994).

Transformation includes any method to deliver the composition of interest to the interior of a cell, encompassing transient and stable transformation of cells with a polynucleotide, transient delivery of a polypeptide, and transient and stable delivery of a microorganism, and the like. Transient transformation is the delivery of a composition of interest to the interior of a cell wherein the composition of interest is not stably inherited by progeny of that cell. Stable transformation is the delivery of a composition of interest to the interior of a cell wherein the composition of interest is stably inherited by progeny of that cell. Transformation frequency is a measure of the number of cells to which the composition of interest has been delivered, and can be measured by a number of standard assays including but not limited to, transient expression of a polynucleotide of interest, transient presence of a polypeptide or other composition of interest, and frequency of stable integration of a polynucleotide of interest into a genome.

This invention can be better understood by reference to the following non-limiting examples. It will be appreciated by those skilled in the art that other embodiments may be practiced without departing from the spirit and the scope of the invention as herein disclosed and claimed. The disclosure of each reference set forth herein is incorporated herein by reference in its entirety.

### Example 1

### A. Compounds and Preparation of Microparticles

A variety of compounds can be tested for their ability to associate a composition of interest with a particle to provide a means to directly deliver the composition of interest to a cell.

i. The cationic liposome, Lipofectin® (InVitrogen, Carlsbad, Calif., USA), can be used to associate nucleic acids to particles. These DNA coated particles can then be delivered to maize cells, using particle bombardment where marker genes are expressed in the host plant cells.

In this example, plasmid DNA (PHP3957) harboring the beta-glucuronidase (GUS) marker gene was associated to 1 μm tungsten particles and bombarded into immature maize embryos. Three days after bombardment, transient expression of GUS was visualized using standard histochemical staining.

Prior to associating the DNA with the particles, the Lipofectin® is diluted 1:1 with sterile distilled water just prior to use. Next, 15 μl of plasmid DNA (0.05 mg/ml) was mixed with 15 μL of diluted Lipofectin®. The DNA and Lipofection® was mixed gently by hand. The DNA:Lipofectin® mixture was added to a tube containing 1 μM tungsten particles and mixed gently by hand until evenly dispersed. Immediately after mixing the DNA:Lipofection®:particle mixture. 10 μL was added to individual macrocarriers that are used for standard helium gun particle bombardment. The macrocarriers containing DNA:Lipofection®:particle mixture were placed on a warm heating plate until the mixture was just dry. Once the DNA:Lipofection®:particle mixture dried, the coated macrocarriers were used to deliver the DNA to immature maize embryos using standard particle bombardment procedures.

Bombarded embryos were incubated at 28° C. in the dark for 3 days on an N6 media containing 20% sucrose, 2,4-D, vitamins and gelrite as the gelling agent. The embryos were then incubated with standard buffer containing X-Gluc to visualize activity of the GUS enzyme. Strong transient expression of the delivered GUS gene was observed in the embryos that were bombarded using this procedure.

In another example immature embryos were co-bombarded with ubi pro::mar::GFPm:pinll (PHP8489) and ubi pro::PAT::pinII (PHP7814), and then split into bialophos selection and no selection groups. Tungsten particles (1 μm) were prepared using CaCl₂+spermidine, TFX-50, or lipofectin. A summary of GFP callus events from three separate bombardments is shown in Table 1.

TABLE 1

| | Bialophos selection | | GFP selection (no bialophos) | |
|---|---|---|---|---|
| Particle Preparation | Event/total | % | Event/total | % |
| CaCl₂ + spermidine | 15/361 | 4.2% | 9/361 | 2.5% |
| TFX-50 | 13/361 | 3.6% | 1/361 | 0.3% |
| Lipofectin | 5/361 | 1.4% | 1/361 | 0.3% |

ii. The cationic lipid TFX-50TH (Promega, Madison, Wis., USA), histone HI (Sigma Chemical Co., St. Louis, Mo., USA), poly-lysine, and PEI (Cat# P3134, Sigma Chemical Co., St. Louis, Mo., USA) were also tested for their ability to associate a polynucleotide or a protein-polynucleotide with a microparticle for particle bombardment. All compounds tested were capable of delivering the composition of interest to a cell.
a. Gold Particles were Prepared with PEI as Follows:
1. Wash particles −1.2 ml 95% ethanol (EtOH) added to 36 mg of 0.6 μm gold particles, vortex 1 minute at high speed, and incubate 15 min at room temperature (RT). Particles are pelleted for 15 min at 4° C., remove supernatant and wash twice with 1.2 ml H2O (vortex 1 min, spin 1 min), wash once with 95% EtOH, then resuspend in final volume of 1.2 ml EtOH (=0.75 mg/25 μl). Washed particles are stored at −20° C.
2. Coat washed gold particles with PEI—centrifuge 25 μl aliquot of washed particles in EtOH, remove supernatant, and resuspend particles in 50 μl DDW. Particles pelleted, supernatant removed, add 22 μl DDW and sonicate particles briefly, add 2.7 μl 1 mM PEI as a droplet on the side of the tube above the gold solution and use pipette to resuspend particles and then incorporate PEI, mix by pipetting 10×. Incubate 10 minutes at RT, optionally with gentle rotation. Sonicate briefly and pipette 3×, flash-freeze in an EtOH/dry ice bath, then lyophilize at least 2 hr.
3. Attach DNA and/or protein to gold/PEI particles—Add 20 μl of 2.5 mM HEPES (pH 7.1) for protein, or 20 μl of DDW for DNA to PEI-coated lyophilized gold particles. Sonicate briefly, pipette 3-10× to mix thoroughly, transfer mixture to clean tube, add 5 ml of protein or DNA solution and quickly pipette 10× (protein 2.5 μg/5 μl in 2.5 mM HEPES, pH 7.1; DNA 1 μg/5 μl in DDW). Incubate 10 min at RT. Pellet particles (10 sec microfuge), remove supernatant, add 60 I EtOH. Particles ready to be spotted on macrocarrier discs.
b. Histone HI

Immature maize embryos were bombarded with single-stranded (ssDNA) or double-stranded DNA (dsDNA) using particles prepared using CaCl₂+spermidine or histone HI. Events were identified, transformation frequency determined, and Southern analysis done on dsDNA events to characterize the copy number and integration loci. A summary of results is presented in Table 2.

TABLE 2

| | | | Southern | | | |
|---|---|---|---|---|---|---|
| | | | | | Integration | |
| | Event/ | | Copy# (%) | | Loci (%) | |
| Treatment | total | % | 1-3 | 4+ | 1-3 | 4+ |
| dsDNA | 49/377 | 12.9% | 10 | 15 | 15 | 11 |
| CaCl₂ + | | | (40%) | (60%) | (57.7%) | (42.3%) |
| spermidine | | | | | | |
| dsDNA HI | 57/368 | 15.5% | 14 | 9 | 16 | 11 |
| | | | (60.9%) | (39.1%) | (59.3%) | (40.7%) |
| ssDNA HI | 1/339 | 0.03% | — | — | — | — |
| ssDNA | 1/352 | 0.03% | — | — | — | — |
| CaCl₂ + | | | | | | |
| spermidine | | | | | | |

### B. Uniformity of Microparticles and Microparticle Suspension

Samples of microparticles were examined by microscopy to determine the level of aggregation and uniformity of coating for untreated, control, and polynucleotide preparations. Aggregation of microparticles reduces the ease of resuspending the particles, and the uniformity of suspension. Extreme clumping may result in more physical force or extreme conditions being used to resuspend prepared particles, possibly damaged the attached composition of interest, for example shearing of DNA.

i. Tungsten 1.8 μm Particles

As observed using scanning electron microscopy, naked tungsten particles, 1.8 μm avg, form small aggregates in the absence of salts and DNA. When treated with CaCl₂-spermidine, aggregates at least as large, typically larger than the naked particles are still observed, but no salt deposits are observed in the aggregates. However, tungsten particles treated with DNA-CaCl₂-spermidine showed a high degree of aggregation, with sticky salt deposits or “bridges” were observed on and between some particles in the aggregates. The level of these deposits varied. Particles treated with DNA-CaCl₂-spermidine were also examined by transmission electron microscopy after negative staining and atomic force microscopy both examinations showed a non-uniform, spotty distribution of the DNA on the particle.

ii. Gold 1.0 μm Particles

As observed using scanning electron microscopy, naked gold particles, 1.0 μm avg, do not aggregate in the absence of salts and DNA, but remain as single particles. Gold particles treated with DNA-TFX-50TH showed very little aggregation of the particles, with the particles generally remaining as single particles. Any aggregates observed were typically composed of only a few particles, approximately 10 or fewer aggregated particles. No deposits or “bridges” were observed in the DNA-TFX-50TH gold particle aggregates examined.

### Example 2

Particles can be prepared to deliver any polynucleotide(s), oligonucleotide(s), polypeptide(s), other compound(s), molecules, and/or microorganism(s), or any combination thereof to plants, plant cells, and/or plant tissues. Particle-based transformation methods are well known and any such method may be used to deliver the prepared particles.

### A. Delivery of Cationic Oligonucleotides

Cationic oligonucleotides can be delivered to plant cells on biolistic particles prepared using a cationic lipid, such as TFX-50TH. Cationic oligonucleotides can be used for targeted modification of a DNA sequence in the genome, see for example U.S. patent publication 2004/0023262 herein incorporated by reference. In one example, plant cells comprising PHP11207 Ubi pro::moPAT::TAG::GFP::pinII were used as the target for cationic oligonucleotides designed to convert the stop codon TAG to TAO (tyrosine) to allow expression of GFP. Four oligonucleotides, as well as controls, were introduced into the callus tissue and/or 10DAP embryos of four different GFP target lines using a biolistic gun method. For microprojectile bombardment gold particles (60 μg/μl) were prepared with the cationic oligonucleotides or plasmid controls (0.1 μg/μl) and TFX-50TH (5 μl of TFX-50TH for 1.0 μg of DNA) then resuspended in 100% ethanol. As shown in Table 3, treatments A-D used different cationic oligos for delivery into the target lines. Treatment E is a negative control, where neither a plasmid nor an oligo was bombarded into GS3 callus. Treatment F is positive control PHP7921 contains Ubi pro::GFP to evaluate particle and/or oligo delivery. All PHP7921 events showed some GFP spots. Treatment G used callus from a stably transformed line of PHP17228 Ubi pro::moPAT::GFP without the TAG stop codon to compare GFP expression in PHP17228 callus material versus induced GFP activation under similar biolistic/culture conditions. PHP17228 material was GFP positive. The bombarded plates containing callus cultures or embryos were screened using Leica DC200 microscope with a GFP2 filter at least twice between day 1 and 10 after bombardment.

Preliminary results indicated that cationic oligos complementary to either transcribed or non-transcribed DNA strand were capable of making the correction that activates GFP. The results indicated that particles treated with either oligonucleotides or plasmids effectively delivered the polynucleotides to the target cells.

TABLE 3

| Treat- | Oligo/Plasmid | | Sel | | |
|---|---|---|---|---|---|
| ment | (0.1 ug/ul) | Loading | media | Pressure | Description |
| A | GMOPHP12 | TFX-50 ™ | 560R | 650 psi | Experiment |
| B | GMOPHP13 | TFX-50 ™ | 560R | 650 psi | Experiment |
| C | GMOPHP17 | TFX-50 ™ | 560R | 650 psi | Experiment |
| D | GMOPHP18 | TFX-50 ™ | 560R | 650 psi | Experiment |
| E | none | TFX-50 ™ | 560R | 650 psi | Control |
| F | PHP7921 | TFX-50 ™ | 560R | 650 psi | Control |
| G | none | TFX-50 ™ | 560R | 650 psi | Control |

### B. Delivery of Recombination Substrate and Transient Protein Expression

Immature embryos from a corn line having a site-specific recombination target site comprising two non-identical recombination sites can be used for subsequent re-transformation with a transfer cassette comprising a polynucleotide of interest flanked by the two non-identical recombination sites using standard particle bombardment methods. The target sites, transfer cassettes, and target lines are created essentially as described in WO 99/25821, herein incorporated by reference. Plasmids comprising the transfer cassette are co-transformed into immature embryos from their respective target lines along with plasmid PHP5096 (Ubi:Ubi-intron::FLPm::pinII). The transfer cassette plasmid is mixed with the FLP-containing plasmid (PHP5096), using 100 ng of the FRT-containing transfer cassette plasmid and 10 ng of the FLP plasmid per bombardment. The FLP plasmid provides transient expression of FLP recombinase without integration of the polynucleotide into the genome of the target cells.

To prepare DNA for delivery, DNA solutions are added to 50 μl of a gold-particle stock solution (0.1 μg/μl of 0.6 μm gold particles). For example, 10 μl of a 0.1 μg/μl solution of transfer cassette plasmid, and 10 μl of a 0.01 μg/μl solution of PHP5096 are first added to 30 μl of water. To this DNA mixture, 50 μl of the gold stock solution is added and the mixture briefly sonicated. Next 5 μl of TFX-50TH (Promega Corp., Madison Wis.) is added and the mixture is placed on a rotary shaker at 100 rpm for 10 minutes. The mixture is briefly centrifuged to pellet the gold particles and remove supernatant. After removal of the excess DNA/TFX solution, 120 μl of absolute EtOH is added, and 10 μl aliquots are dispensed onto the macrocarriers typically used with the DuPont PDS-1000 Helium Particle Gun. The gold particles with adhered DNA are allowed to dry onto the carriers and then these are used for standard particle bombardment. After re-transformation the immature embryos are placed onto 560P medium for two weeks to recover, and then moved to selection medium, to identify re-transformation events which are subsequently regenerated into plants using standard methods.

### C. Delivery of Agrobacterium

Microorganisms, such as bacteria, bacteria comprising a bacteriophage, or a virus can be delivered via direct delivery of microparticles comprising the microorganism. For example, Agrobacterium comprising a T-DNA which comprises a polynucleotide of interest have been adhered to microparticles for delivery to plant cell by particle bombardment, producing a fertile transgenic plant comprising the polynucleotide of interest stably incorporated in its genome, see for example U.S. Pat. No. 5,932,782, herein incorporated by reference.

In Bidney (U.S. Pat. No. 5,932,782), Agrobacterium comprising a T-DNA were grown in standard media to various cell densities, mixed with gold particles, applied to macroprojectiles, and dried for varying times. These preparations were used to bombard plant tissues.

Various association agents can be tested for their effect on microorganism growth and/or viability, concentration, temperature of preparation, appropriate microorganism cell density, plant cell transformation frequency and/or stability. For example, Agrobacterium cultures comprising a T-DNA with a polynucleotide of interest, such as a visible marker and/or selectable marker, can be growth in standard media to densities of 0.5-2.0 OD₆₀₀, and aliquots of the cultures mixed with varying concentrations of TFX-50™. This is mixed with a fixed quantity of gold particles, and aliquots applied to macrocarriers and dried for varying amounts of time. The macrocarriers are used to bombard a plant tissue, such as immature maize embryos prepared using standard conditions. Various microparticle compositions, as well as microparticle sizes can be tested as well. Bombarded tissue is regenerated under standard conditions and screened for the polynucleotide(s) of interest. Bacterial media plates can be bombarded to determine Agrobacterium viability under the experimental conditions.

### Example 3

A number of association agents can be used to bind a polynucleotide or a protein-polynucleotide with a microparticle for particle bombardment. In this example, PEI is tested for delivery of polynucleotides and co-delivery of polynucleotides and polypeptides.

Uncoated gold particles, and gold particles coated with PEI were used. PEI reagent was received as a 50% w/v aqueous solution and used to make a 1M stock as follows: 1.85 g of PEI was mixed with 5.0 ml sterile deionize water, 1.0 ml 10N HCl, the pH of this solution was measured with a disposable pH indicator strip which indicated a pH of approximately 7.0, this solution was brought to a final volume of 10 ml with sterile deionized water to produce the 1M PEI stock. The concentration is based on the monomer equivalents of PEI. This 1M stock was further diluted with deionized water to produce a 0.54 mM working stock, which was used to prepare 1.6 μm gold particles as follows: 20.25 mg colloidal gold particles were washed with 675 μl 95% ethanol, pelleted, rinsed with 100 μl deionized water, pelleted, and resuspended in 540 μl deionized water; 20 μl aliquots (0.75 mg gold) are resuspended, and 5 μl of 0.54 mM PEI added and this suspension mixed for 10 minutes by vortexing; this mixture is flash frozen, then lyophilized to remove water.

The following plasmids and polypeptides were prepared:

PHP1654 comprises GAL4 upstream activating sequence::CaMV35S promoter::Ω5′UTR::ADH1 intron::Luciferase

PHP1209 comprises CaMV35S promoter::GAL4-VP66

GAL4-VP16 fusion protein, M.W. 26.5 kDa, was purchased from Trevigen (Gaithersburg, Md., USA) and a working stock at 1.8 μg/μl used.

The polynucleotides and polynucleotide/polypeptide compositions were bound to the gold particles. PEI-coated particles were sonicated, then 22.2 μl 5 mM HEPES (pH 7.1) and 0.9-1.0 μl plasmid, and optionally 1.4 μl Gal4-VP16 protein added and the mixture incubated for 10 minutes at room temperature. The mixture was pelleted, and the pellet resuspended in 60 μl ethanol for use. Uncoated particles were sonicated, then 2.2 μl 250 mM HEPES (pH 7.1) and 0.9-1.0 μl plasmid, and optionally 1.4 μl Gal4-VP16 protein added and the mixture resuspended by pipetting. Particles with plasmid only were flash frozen, lyophilized, and resuspended in 60 μl ethanol for use. Particles with plasmid and protein were incubated 15 minutes at 37° C., then flash frozen, lyophilized, and resuspended in 60 μl ethanol for use.

Maize suspension callus cells were pelleted, resuspended in osmoticum, and plated onto pre-wetted paper filter disks for bombardment. Cells were bombarded once per plate using 10 μl of prepared gold particle suspension per macrocarrier disk in a biolistic helium gun using 650 psi rupture disk. Three replicate plates were bombarded for each particle preparation. Cells were incubated overnight at 27° C. before assaying for luciferase reporter gene expression. Transient expression results for luciferase activity (net LUC light units/g soluble protein) are shown in Table 4.

TABLE 4

Luciferase

| Particle | Composition | Mean | Std. Dev. |
|---|---|---|---|
| none | Untreated cell control | 0 | 0.2 |
| PEI | PHP1654 | 10.08 | 10.47 |
| PEI | PHP1209 + PHP1654 | 1904.06 | 382.62 |
| PEI | Gal4-VP16 + PHP1654 | 1721.26 | 513.09 |
| Uncoated | PHP1654 | 7.91 | 3.76 |
| Uncoated | PHP1209 + PHP1654 | 2615.94 | 1036.46 |
| Uncoated | Gal4-VP16 + PHP1654 | 301.93 | 153.73 |

### Example 4

Nanoparticle:microparticle mixtures can be prepared using any suitable nanoparticle, any suitable microparticle, and any combinations thereof. The composition(s) of interest can be associated with distinct types of particles separately, sequentially, simultaneously, or any combinations thereof.

Any suitable nanoparticle and appropriate preparation and loading system can be used. For example, for delivery of a composition of interest into a plant cell by bombardment, capped mesoporous silica nanoparticles (MSNs) can be prepared as described in Torney et al. (2007) Nat Nanotechnol 2:295. As described by Torney and colleagues, a Bio-Rad Biolistic PDS-1000/He particle delivery system was used for MSN delivery into plant cells. DNA was coated onto gold-capped MSNs, using a standard protocol for the biolistics (Sanford et al. (1993) Methods Enzymol. 217: 483-509) with modifications to prevent the agglomeration.

To facilitate particle gun delivery of the nanoparticles into plant cells, the prepared nanoparticles can be combined with microparticles using any of the methods described in the previous examples. The nanoparticles can be associated with microparticle to form a uniform association of nano and microparticles.

For example, 0.6 μm (average diameter) gold pellets can be associated with prepared and loaded nanoparticles using a water-soluble cationic lipid Tfx™-50 (Promega, Madison, Wis.) as follows: prepare and load the nanoparticles with a composition of interest to produce a loaded nanoparticle such as a MSN loaded with a DNA and optionally capped. To the prepared nanoparticles, for example 10 μg prepared nanoparticles, add 20 μl washed gold particles (15 mg/ml) in water; 10 μl Tfx-50 in water; and mix carefully.

Once combined, the mixture can be treated similarly to a microparticle preparation. For example, this mixture can be stored on ice during preparation of macrocarriers, typically about 10 min. Pellet the particle mixture in a microfuge at 10,000 rpm for 1 min, remove supernatant. Carefully rinse the pellet with 100 ml of 100% EtOH without resuspending the pellet, carefully remove the EtOH rinse. Add 20 μl of 100% EtOH and carefully resuspend the particles by brief sonication, 10 μl spotted onto the center of each macrocarrier and allowed to dry about 2 minutes before bombardment.

It is further expected that the nanoparticle-microparticle mixture will behave similar to a microparticle preparation for purposes of delivery of the composition of interest into a plant cell. Therefore, standard particle gun delivery parameters for microparticles can be used to delivery the nanoparticle-microparticle mixture.

For example, sample plates of maize target embryos can be bombarded twice per plate using approximately 0.5 μg of DNA per shot using the Bio-Rad PDS-1000/He device (Bio-Rad Laboratories, Hercules, Calif.) with a rupture pressure of 450 PSI, a vacuum pressure of 27-28 inches of Hg, and a particle flight distance of 8.5 cm.

Transgenic soybean lines are generated by particle gun bombardment (Klein et al. Nature 327:70-73 (1987); U.S. Pat. No. 4,945,050) using a BIORAD Biolistic PDS1000/He instrument. Soybean embryogenic suspension cultures are initiated and maintained in culture. Approximately 150 to 250 mg of two-week-old suspension culture is placed in a petri plate and the residual liquid removed using a pipette. For every seventeen bombardment transformations, 85 μL of particle suspension is prepared containing 1 to 90 picograms (pg) of DNA/bp of each DNA. The DNAs in suspension are added to 50 μL of a 10-60 mg/mL 0.6 μm gold particle suspension, mixed, and 5 μl of TFX-50™ is added. The mixture is vortexed, microfuged for 5 sec, and the supernatant removed. The DNA coated particles are then washed once with 150 μL of 100% ethanol, vortexed and pelleted, then resuspended in 85 μL of anhydrous ethanol. Five μL of the DNA coated gold particles are then loaded on each macrocarrier disk. The tissue is placed about 3.5 inches away from the retaining screen and each plate of tissue is bombarded once. Membrane rupture pressure is set at 650 psi and the chamber is evacuated to −28 inches of Hg. Following bombardment, the tissue from each plate is divided between two flasks, placed back into liquid media, and cultured.

In some examples, the microparticles and/or other nanoparticles may be loaded with further compositions of interest prior to, during, or after combination with the nanoparticles. For example, a DNA composition of interest may be loaded onto the microparticles using any standard method, and/or any methods described in the previous Examples. Further, the nanoparticle-microparticle mixture may be loaded with further compositions of interest during or after the initial association step.

For example, a protein or mRNA, such as a recombinase or a homing endonuclease, can be loaded into the nanoparticle pores. Optionally, the pores can be capped. Then, a transfer cassette comprising a polynucleotide of interest to be inserted at a target site can be added to the loaded nanoparticles, optionally in the presence of gold or tungsten microparticles, and TFX-50™ added to associate the transfer cassette to the particles, and associate the nanoparticles with the microparticles. The nanoparticle:microparticle mixture is delivered to plant cells using appropriate standard bombardment conditions. If the pores were capped, the caps are removed and the mRNA or protein released and is transiently available to effect integration of the polynucleotide of interest at the target site.

For example, a cell growth stimulating factor, such as a BBM and/or WUS mRNA or protein, can be loaded into the nanoparticle pores. Optionally, the pores can be capped. A DNA construct comprising a polynucleotide of interest to be inserted in a genome can be added to the loaded nanoparticles, optionally in the presence of gold or tungsten microparticles, and TFX-50™ added to associate the transfer cassette to the particles, and associate the nanoparticles with the microparticles. The nanoparticle:microparticle mixture is delivered to plant cells using appropriate standard bombardment conditions. If the pores were capped, the caps are removed and the mRNA or protein released and is transiently available to enhance transformation frequency in the plant cells, without having integration of the factor into the target genome.

### Example 5

Any method of plastid transformation and/or vector design can be used to transform a host plant cell. In many cases, the chloroplast transformation method essentially as described by Svab & Maliga (Proc Natl Acad Sci 90:913-917 (1993)) is used.

For example, a plasmid vector carrying a chimeric aadA gene is operably linked to a 16S rRNA promoter region (Prrn) and psbA 3′ region. This cassette is located between the regions of homology to the tobacco rbcL gene and ORF512.

Tobacco (N. tabacum) plants are grown aseptically on agar-solidified medium containing MS salts and sucrose (30 g/liter). Leaves are placed abaxial side up on RMOP medium for bombardment. Tungsten microprojectiles (1 μm) are coated with the plasmid vector, and bombardment performed with the DuPont PDS1000. Spectinomycin-resistant calli and shoots are selected on RMOP medium containing 500 μg/ml spectinomycin. Resistant shoots are regenerated on the same selective medium and rooted on MS agar to obtain TO plants.

Nanoparticles have been used to deliver plasmid DNA to a variety of animal cells. It has been found that when DNA coated nanoparticles are incubated with cells lacking a cell wall, the cells take up the particles and begin expressing any product encoded by the DNA. Using the nanoparticle:microparticle mixture, the chloroplast transformation vector can be associated with the nanoparticles. Optionally, the microparticles and/or the nanoparticles may further comprise other compositions of interest that may enhance cell growth, chloroplast transformation, integration, targeting, selection, homotransplastomicity, or any other step in the process of generating and recovering transplastomic plant cells, plants, and/or seeds. For example, a chloroplast targeting sequence can be associated with the nanoparticles to facilitate nanoparticle targeting and/or update. Optionally, a chemicals the reduce cell stress response to bombardment could be provided, such as chemicals that inhibit ethylene action. Several of these chemicals are hazardous, and therefore can be loaded into nanoparticle pores or layers which are then capped or encapsylated. This provides a means to reduce human exposure to hazardous compounds. These nanoparticles may be associated with the plasmid, or may be added directly to the mixture with plasmid-coated nanoparticles and microparticles.

Although the foregoing invention has been described in some detail by way of illustration and example for purposes of clarity of understanding, certain changes, modifications, and derivations may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

What is claimed is:

1. A method of delivering a composition of interest to a plant cell, the method comprising bombarding a nanoparticle:microparticle mixture comprising of at least one nanoparticle associated with a microparticle via a lipid compound, wherein the nanoparticle comprises the composition of interest.

2. The method of claim 1, wherein the transformation frequency is increased as compared to a method that does not use a microparticle.

3. The method of claim 1 or claim 2, wherein the transformation frequency is increased as compared to a method that does not use a lipid compound.

4. The method of any one of claims 1-3, wherein the transformation frequency is increased as compared to a method that does not use the composition of interest.

5. The method of any one of claims 1-4 wherein the composition of interest comprises an organic substance, an inorganic substance, a drug, a hormone, a hormone antagonist, a ligand, an inducer, a polynucleotide, a polypeptide, a microorganism, a subcellular organelle, a growth factor, a polysaccharide, a vitamin, a messenger, a co-factor, or any combination thereof.

6. The method of claim 5 wherein the composition of interest is selected from the group consisting of an auxin, indole acetic acid, naphthalene acetic acid, dicamba, 2,4-D, an anti-auxin, 2,4,6-trichlorobenzoic acid, 2-(2,4-dichlorophenoxy) proprionic acid a gibberellin, a cytokinin, zeatin, kinetin, thidiazuron, benzylaminopurin, an abscisic acid, an ABA antagonist, aminotriazole, ethylene, 1-propene, an ethylene antagonist, 1-butene, 1-pentene, 1-hexane, 1-octene, 1-decene, 1-dodecene, an ethylene blocker, and norbornadiene.

7. The method of any one of claims 1-6, wherein the composition of interest comprises a compound that enhances transformation frequency.

8. The method of claim 7, wherein the compound is a hormone, a hormone antagonist, a polynucleotide that stimulates cell growth, or a polypeptide that stimulates cell growth.

9. The method of claim 7, wherein the composition of interest comprises an auxin or a cytokinin.

10. The method of claim 8, wherein the composition of interest comprises a polynucleotide encoding a polypeptide that stimulates cell growth selected from the group consisting of a knotted polypeptide, a babyboom polypeptide, a wuschel polypeptide, a cell cycle polypeptide, and any combinations thereof.

11. The method of claim 1, wherein the composition of interest comprises a polynucleotide of interest, wherein the polynucleotide is integrated at a target site in a plant cell genome.

12. The method of any one of claims 1-11, wherein the composition of interest is delivered to a plastid.

13. The method of claim 12, wherein the composition of interest comprises a polynucleotide of interest, wherein the polynucleotide of interest is integrated in the plastid genome.

14. The method of claim 5 wherein the composition of interest comprises a ligand that induces or de-represses a chemically-inducible expression cassette.

15. The method of claim 14, wherein the ligand is a tetracycline or a tetracycline analogue.

16. The method of claim 5 wherein the composition of interest comprises a polynucleotide encoding a polypeptide that confers an agronomic trait, a disease resistance trait, a insect resistance trait, a herbicide tolerance trait, herbicide resistance, antibiotic resistance, chemical resistance, efficient nitrogen use, nutritional enhancement, an oil trait, a starch trait, a protein trait, a commercial processing trait, a feed trait, male sterility, improved rate of growth, or any combinations thereof.

17. The method of claim 8 wherein the composition of interest comprises a polynucleotide that suppresses expression of a target molecule, wherein the polynucleotide is selected from the group consisting of a double-stranded RNA, miRNA precursor, a miRNA, a sRNA precursor, a sRNA, a transacting sRNA precursor, a transacting sRNA, an RNAi precursor, an antisense polynucleotide precursor, an antisense polynucleotide, a sense-suppression precursor, a sense-suppression polynucleotide, and a ribozyme.

18. The method of claim 5 wherein the composition of interest is a polynucleotide encoding a polypeptide selected from the group consisting of a recombinase, an integrase, a site-specific recombinase, a homing endonuclease, a transposase, a meganuclease, a DNA polymerase, a DNA ligase, and a restriction enzyme.

19. The method of claim 18, wherein the frequency of gene targeting is increased as compared to a method that does not include the composition of interest.

20. The method of claim 5 wherein encoded polypeptide is a selectable marker selected from the group consisting of a fluorescent protein and a luciferase protein.

21. The method of any one of claims 1-20, further comprising a second composition of interest associated with the particle mixture.

22. The method of claim 21 wherein the composition of interest is different than the second composition of interest.

23. The method of claim 21 or 22, wherein the second composition of interest is associated with the microparticle or the nanoparticle of the particle mixture.

24. The method of any one of claims 21-23, wherein the composition of interest is a chemical inducer and the second composition of interest is a polynucleotide operably linked to a promoter chemically inducible by the composition of interest.

25. The method of any one of claims 21-24, wherein the mixture comprises the second composition of interest associated with the microparticle or the nanoparticle, wherein the release of the composition of interest is delayed, timed, sequential or any combination thereof as compared to the release of the second composition of interest.

26. The method of claims 21-25, wherein the second composition of interest comprises an organic substance, an inorganic substance, a drug, a hormone, a hormone antagonist, a ligand, an inducer, a polynucleotide, a polypeptide, a microorganism, a subcellular organelle, a growth factor, a polysaccharide, a vitamin, a messenger, a co-factor, or any combinations thereof.

27. The method of any one of claims 21-26, wherein the second composition of interest is associated with the microparticle, and wherein the second composition of interest does not substantially dissociate from the microparticle after delivery to the plant cell.

28. The method of any one of claims 1-27, wherein the nanoparticle comprises a ceramic nanoparticle, a gold nanoparticle, a gold-coated nanoparticle, a porous nanoparticle, a mesoporous nanoparticle, a silica nanoparticle, a mesoporous silica nanoparticle, a polymer nanoparticle, a tungsten nanoparticle, a gelatin nanoparticle, metal nanoparticles, metal oxide nanoparticles, metal-nonoxide nanoparticles, organic nanoparticles, biomolecular nanoparticles, a nanoshell, a nanocore, a nanosphere, a nanorod, a magnetic nanoparticle, a nanocrystal, or any combinations thereof.

29. The method of any one of claims 1-28, wherein the nanoparticle comprises polymers selected from the group consisting of ceramic, hydrolysed silane, silica, polysilsesquioxane polylactide, polyglycolide, poly lactic glycolic acids (PLGA), poly aminoacids, polyaminoacids, glycosamino glycans, lipidated glycosaminoglycans, any combinations thereof and copolymers thereof.

30. The method of any one of claims 1-29, wherein the composition of interest is associated with at least one region of the nanoparticle selected from the group consisting of a core, a layer, and a pore.

31. The method of any one of claims 1-30 wherein the composition of interest is encapsulated in the core or within the layer or any combinations thereof.

32. The method of any one of claims 1-31 wherein the composition of interest is incorporated into the core or the layer or any combinations thereof.

33. The method of any one of claims 1-32 wherein the composition of interest is associated with a surface of the core, a surface of the layer, a surface of the pore or any combinations thereof.

34. The method of claim 33, wherein the association between the composition of interest and any surface is via chemical linking, physical linking, adsorption, chemical conjugation, or any combinations thereof.

35. The method of claim 33 or 34, wherein the association between the composition of interest and any surface is via a chemical bond selected the group consisting of a covalent bond, a non-covalent bond, an ionic bond, a metallic bond, a hydrogen bond, or any combinations thereof.

36. The method of any one of claims 33-35, wherein the composition of interest is chemically linked to the surface via a functional group selected from the group consisting of an amide, an amine, a thiol, a carboxyl group, or any combinations thereof.

37. The method of any one of claims 1-33 wherein the composition of interest is adsorbed to the surface via a lipid compound selected from the group consisting of a lipid solution, a cationic lipid solution, a liposome solution, and any combinations thereof.

38. The method of claim 37 wherein the lipid compound comprises a cationic lipid solution selected from the group consisting of N,N,N′,N′-tetramethyl-N,N′-bis(2-hydroxylethyl)-2,3-di(oleoyloxy)-1,4-butanediammonium iodide, and L-dioleoyl phosphatidylethanolamine (DOPE).

39. The method of claim 37 or 38 wherein the lipid compound is selected from the group consisting of Tfx-10™, Tfx-20™, Tfx-50™, Lipofectin™, Lipofectamine™, Cellfectin™, Effectene™, Cytofectin GSV™, Perfect Lipids™, DOTAP™, DMRIE-C™, FuGENE-6™, Superfect™, Polyfect™, and any combinations thereof.

40. The method of any one of claims 1-39 wherein the composition of interest is released from the nanoparticle by dissociation, degradation, cleavage, or diffusion from the nanoparticle or combinations thereof.

41. The method of any one of claims 1-40, wherein the release of the composition of interest from a pore is inhibited by a cap covering the pore.

42. The method of claim 41, wherein the cap comprises a nanoparticle, a nanosphere, a nanorod, an inorganic particle, an inorganic molecule, an organic molecule, an oligomer, a polymer, dendrimers, a polypeptide, a protein, an oligonucleotide, an oligosaccharide, a polysaccharide, or any combinations thereof.

43. The method of any one of claims 1-42, wherein the composition of interest is released from a pore by removal of a cap.

44. The method of any one of claims 1-43, wherein the composition of interest is released from the surface of a core, a layer, or a pore by disruption of a chemical bond selected from the group consisting of a covalent bond, a non-covalent bond, an ionic bond, a metallic bond, a hydrogen bond, and any combinations thereof.

45. The method of any one of claims 1-44 wherein the composition of interest is released from the surface of a core, a layer, or a pore by disruption of the functional group selected from the group consisting of an amide, an ester, an amine, a thiol, a carboxyl group, and any combinations thereof.

46. The method of any one of claims 1-45, wherein the microparticle comprises gold, tungsten, palladium, rhodium, platinum, iridium, silica, whiskers or any combinations thereof.

47. The method of any one of claims 1-46, wherein the nanoparticle is non-covalently associated to the microparticle.

48. The method of any one of claims 1-47, wherein the plant cell is from a monocotyledonous or a dicotyledonous plant.

49. The method of claim 48, wherein the plant cell is selected from the group consisting of maize, rice, wheat, barley, millet, sorghum, rye, soybean, alfalfa, canola, Arabidopsis, tobacco, sunflower, cotton, sugarcane, and safflower.

50. The method of any one of claims 1-49, further comprising selecting a plant cell comprising a polynucleotide of interest stably incorporated into a genome of the plant cell.

51. The method of claim 50, further comprising regenerating a plant from the stably transformed plant cell, wherein the plant comprises the polynucleotide of interest.

52. A stably transformed plant cell produced by method of any one of claims 1-48.

53. A method of preparing a mixture of particles for direct delivery of a composition of interest to a plant cell comprising associating at least one nanoparticle comprising the composition of interest with a microparticle via a lipid compound.

54. The method of claim 53, wherein the lipid compound is selected from the group consisting of a lipid solution, a cationic lipid solution, a liposome solution, and any combinations thereof.

55. The method of claim 54, wherein the composition of interest is attached to the nanoparticle or the microparticle via a lipid compound selected from the group consisting of a lipid compound a cationic lipid solution, a liposome solution, and any combinations thereof.

56. The method of any one of claims 53-55, wherein the lipid compound is a cationic lipid solution comprising N,N,N′,N′-tetramethyl-N,N′-bis(2-hydroxylethyl)-2,3-di(oleoyloxy)-1,4-butanediammonium iodide.

57. The method of any one of claims 53-56, wherein the cationic lipid solution comprises L-dioleoyl phosphatidylethanolamine (DOPE).

58. The method of any one of claims 53-55, wherein the lipid compound is selected from the group consisting of Tfx-10™, Tfx-20™, Tfx-50™, Lipofectin™, Lipofectamine™, Cellfectin™, Effectene™, Cytofectin GSV™, Perfect Lipids™, DOTAP™, DMRIE-C™, FuGENE-6™, Superfect™, and Polyfect™.

59. The method of any one of claims 53-58, wherein the nanoparticle is associated with the microparticle via liposome encapsulation.

60. A mixture of particles produced by the method of any one of claims 53-59.

61. A mixture of particles for direct delivery of a composition of interest, the mixture comprising of at least one nanoparticle associated with a microparticle via a lipid compound, wherein the nanoparticle comprises the composition of interest and wherein the lipid compound is selected from the group consisting of a lipid solution, a cationic lipid solution, a liposome solution, and any combinations thereof.

62. The mixture of claim 61, wherein the composition of interest is attached to the nanoparticle or the microparticle via a lipid compound selected from the group consisting of a lipid solution, a cationic lipid solution, a liposome solution, and any combinations thereof.

63. The mixture of claim 61 or 62, wherein the lipid compound is a cationic lipid solution comprising N,N,N′,N′-tetramethyl-N,N′-bis(2-hydroxylethyl)-2,3-di(oleoyloxy)-1,4-butanediammonium iodide.

64. The mixture of any one of claims 61-63, wherein the cationic lipid solution comprises L-dioleoyl phosphatidylethanolamine (DOPE).

65. The mixture of claim 61 or 62, wherein the lipid compound is selected from the group consisting of Tfx-10™, Tfx-20™, Tfx-50™, Lipofectin™, Lipofectamine™, Cellfectin™, Effectene™, Cytofectin GSV™, Perfect Lipids™, DOTAP™, DMRIE-C™, FuGENE-6™, Superfect™, and Polyfect™.

66. The mixture of any one of claims 61-65, wherein the nanoparticle is associated with the microparticle via liposome encapsulation.

67. The mixture of any one of claims 61-66, wherein the composition of interest is selected from the group consisting of an organic substance, an inorganic substance, a drug, a hormone, a hormone antagonist, a ligand, an inducer, a polynucleotide, a polypeptide, a microorganism, or a subcellular organelle, growth factor, polysaccharide, a vitamin, a messenger, a co-factor, and any combinations thereof.
